(19) United States
(12) Patent Application Publication
(10) Pub. No.: US 2025/0064401 A1
(43) Pub. Date: Feb. 27, 2025

(54) MEASURING STATION WITH SWEAT ACTIVITY MEASUREMENT

(71) Applicant: Withings, ISSY LES MOULINEAUX (FR)

(72) Inventors: Thomas KRAUSE, ISSY LES MOULINEAUX (FR); Antoine JOUSSAIN, ISSY LES MOULINEAUX (FR); Jean-Louis WANG, ISSY LES MOULINEAUX (FR); Jérôme BREDIN, ISSY LES MOULINEAUX (FR); Roger YU, ISSY LES MOULINEAUX (FR); Brice BRAC DE LA PERRIÈRE, ISSY LES MOULINEAUX (FR); Victorine CALLENS, ISSY LES MOULINEAUX (FR)

(21) Appl. No.: 18/725,606

(22) PCT Filed: Dec. 16, 2022
(86) PCT No.: PCT/EP2022/086492

§ 371 Date: Jun. 28, 2024

(30) Foreign Application Priority Data
Dec. 31, 2021 (FR) 2114739

(51) Int. Cl. A61B 5/00; A61B 5/0531; A61B 5/0535; A61B 5/318

(52) U.S. Cl. CPC A61B 5/6892; A61B 5/0531; A61B 5/0535; A61B 5/318; A61B 5/4266; A61B 5/6825; A61B 5/6829; A61B 2560/0468

(57) ABSTRACT

A measuring station includes a left set of electrodes, including at least two electrically independent electrodes arranged to contact the underside of a user's left foot, a right set of electrodes, including at least two electrically independent electrodes arranged to contact the underside of a user's right foot, a direct voltage source, a switch configured to activate and deactivate a DC configuration in which at least one electrode of at least one set of electrodes is connected to the direct voltage source.

### FIELD

The present description relates to the monitoring of a user's health, and more specifically to measuring stations for implementing one or more measurements of a user's biometric signals (or physiological parameters).

At least one of the following data is determined by the measuring station of the present application: weight or mass, electrocardiogram (ECG), impedance measurement (impedance analysis of the human body), including impedance-plethysmogram (IPG), impedance-cardiogram (ICG) and bioimpedance analysis (BIA, for the mass of fat, water, muscle, etc.), photoplethysmogram (PPG), ballistocardiogram (BCG), electrochemical skin conductance analysis (“ESC analysis” or simply “ESC” in the present description) and evaluation of sweat function (sometimes called “sudogram” in the present application), heart rate (“HR”), pulse wave velocity (“PWV”), and so on.

### BACKGROUND

Document WO2010/122252, from 2010, describes a connected scale with weight and bioimpedance measurement. Documents EP3087914 and EP3095380, from 2015, describe a connected scale for obtaining information on the user's cardiovascular condition, including pulse transit time (PTT) measurement using BCG and IPG. Document WO2021/164561 describes a scale with handle for weight measurement, multi-channel ECG and segmental BIA.

The development of new measuring devices that can be used in the home is desirable.

### SUMMARY

The aim of the present description is to provide a measuring station for obtaining various biometric signal measurements with improved quality.

The invention is defined in the claims.

In an embodiment, the description presents a measuring station comprising:
a left set of electrodes, comprising at least two electrically independent electrodes arranged to contact the underside of a user's left foot,
a right set of electrodes, comprising at least two electrically independent electrodes arranged to contact the underside of a user's right foot,
a direct voltage source,
a switch configured to activate and deactivate a direct current, DC, configuration in which at least one electrode, called active electrode, of at least one set of electrodes is connected to the direct voltage source.

In particular, an electrode of each set is connected to the direct voltage source (at its two opposite terminals). One set operates as a cathode, the other as an anode.

In an embodiment, in the DC configuration, at least one electrode, known as the passive electrode, of a set of electrodes is not connected to the direct voltage source. The passive electrode may be a high-impedance connected electrode, for example greater than 500 kOhm. In a variant, at least one electrode of each set of electrodes is not connected to the direct voltage source.

In an embodiment, the measuring station further comprises an alternating current source, and the switch is configured to selectively activate or deactivate an alternating current configuration, so-called AC configuration (ACs, ACf, ACb), in which at least one electrode of the left set of electrodes and the right set of electrodes is connected to the alternating current source. The switch is configured to switch at least between the DC configuration and the AC configuration. DC and AC configurations cannot be activated simultaneously. The AC configuration enables BIA (between the legs, and segmental with a handle), IPG between the legs and IPG in the foot.

In an embodiment, in an AC configuration (ACf, ACb), the switch is configured to connect at least two electrodes of the left set of electrodes and the right set of electrodes to the terminals of the alternating current source. This arrangement enables BIA between the legs, IPG between the legs and IPG in the foot. The switch is further configured to connect at least two electrodes of the left set of electrodes and the right set of electrodes to the terminals of a voltmeter.

In an embodiment, in an AC configuration (ACb), the switch is configured to connect at least one electrode of the left set and at least one electrode of the right set to the terminals of the alternating current source. This arrangement enables BIA between the legs and IPG between the legs. The switch is further configured to connect at least one electrode of the left set and at least one electrode of the right set to the voltmeter terminals.

In an embodiment, in AC configuration (ACf), the switch is configured to connect at least one electrode of a set to one terminal of the current source and another electrode of the same set to another terminal of the current source. This arrangement enables IPG to be performed in the foot. The switch is further configured to connect at least two electrodes of the same set to the terminals of a voltmeter. In particular, the two electrodes connected to the voltmeter are not adjacent.

In an embodiment, the left set (LG) and/or the right set (RG) each comprise three electrically independent electrodes, and, in DC configuration, the switch is configured to connect at least two electrodes of the left set (LG) and/or at least two electrodes of the right set (RG) to the direct voltage source.

In an embodiment, the left set (LG) and/or the right set (RG) each comprise four electrically independent electrodes, and, in DC configuration, the switch is configured to connect at least three electrodes of the left set and/or at least three electrodes of the right set to the direct voltage source.

In an embodiment, the left set (LG) and/or the right set (RG) each comprise five electrically independent electrodes, and, in DC configuration, the switch is configured to connect at least four electrodes of the left set (LG) and/or at least four electrodes of the right set (RG) to the direct voltage source.

In an embodiment, the left set (LG) and/or the right set (RD) each comprise at least six electrically independent electrodes, and, in DC configuration, the switch is configured to connect at least five electrodes of the left set (LG) and/or at least five electrodes of the right set (RD) to the direct voltage source. In AC configuration, at least two inactive electrodes may be located between the two electrodes connected to the voltmeter. One of these inactive electrodes in AC configuration may be the passive electrode in DC configuration.

In an embodiment, the left set (LG) and/or the right set (RG) each comprise at least seven electrically independent electrodes, and, in DC configuration, the switch is configured to connect at least six electrodes of the left set (LG) and/or at least six electrodes of the right set (RG) to the direct voltage source. In AC configuration, at least three inactive electrodes may be located between the two electrodes connected to the voltmeter, one of which may be the passive electrode in DC configuration.

In a DC configuration, the passive electrode is located between two active electrodes.

In an embodiment, the electrodes are arranged side by side, spaced apart, along the length of the measuring station.

In an embodiment, the electrodes are in the form of parallel strips.

In an embodiment, the electrodes are spaced apart along the length of the measuring station. This limits the effects of front/back foot placement. In an embodiment, the end electrodes along the length are wider, to limit the effects of lengthwise foot placement.

In an embodiment, electrodes configured to be active in the DC configuration have a dimension in the direction of a measuring station length that is equal to or greater than that of electrodes configured to be passive in the DC configuration.

In an embodiment, for a right or left set of electrodes, the electrodes configured to be active in the DC configuration cover at least 50% of the surface area (Sa) of the convex envelope (Se) defined by all the electrodes in the set. Alternatively or additionally, the distance (Dmax) between the end electrodes, including the electrodes, along a length of the measuring station is at least 20 cm. Alternatively or additionally, the electrodes extend over a width of the station by at least 10 cm, or even at least 15 cm. This limits the effects of lateral foot placement.

In an embodiment, the electrodes of the left set extend along a width of the measuring station over more than 40% of the width of the measuring station, and the electrodes of the right set extend along a width of the measuring station over more than 40% of the width of the measuring station.

In an embodiment, the electrodes comprise an indium tin oxide, ITO, material.

In an embodiment, the direct voltage source is configured to selectively apply successive steps of constant voltage to a pair of so-called active electrodes, said electrodes of the pair constituting an anode and a cathode. The voltage values of the successive steps may be decreasing (i.e. each step has a lower voltage value than the previous step) and/or last between 500 ms and 2 s each.

In an embodiment, the measuring station according to any of the preceding claims further comprises a weight sensor.

In an embodiment, the measuring station comprises a base including the left set of electrodes and the right set of electrodes, and comprises a handle with at least one electrode suitable for contact with the hand. In DC configuration, the switch is configured to connect the handle electrode to a high impedance. In particular, the switch may be configured to connect all the base electrodes to the terminals of the direct voltage source.

In an embodiment, the left and right set electrodes comprise an indium tin oxide, ITO, material.

The description also presents a measuring station comprising:
at least one pair of electrodes comprising an indium tin oxide, ITO, material,
a direct voltage source configured to selectively apply direct voltage steps to the pair of electrodes, referred to as active electrodes, said electrodes of the pair constituting an anode and a cathode. This measuring station may perform an ESC.

In an embodiment, the description also presents a measuring station comprising:
a base able to receive at least one foot of a user,
a handle suitable for receiving at least one hand of a user,
a cable connecting the base to the handle,
   wherein the handle comprises a handle master switch configured to alternate between an electrocardiogram position and an impedance measurement position, and the base comprises a master switch configured to alternate between an electrocardiogram position and an impedance measurement position.

In an embodiment, the handle comprises four electrodes, each electrode being connected to a switch for connecting said electrode to an electrocardiogram electrical circuit or to an impedance measurement circuit, the ECG electrical circuit and the impedance measurement circuit being connected to the master switch. The four electrodes enable the handle to perform BIA (especially segmental) and ECG measurements.

In an embodiment, the electrocardiogram electrical circuit and the impedance measurement electrical circuit enable electrical signals to be processed before they pass through the cable.

The description also presents a method of measurement (e.g. ESC acquisition) using a station as described above, the method comprising a step of switching, using the switch, between two different configurations.

### BRIEF DESCRIPTION OF THE DRAWINGS

The following figures illustrate the elements described in this description.

FIG. 1: FIG. 1 shows a three-dimensional view of a measuring station with a handle, according to an embodiment;

FIG. 2: FIG. 2 shows a side view of the station shown in FIG. 1;

FIG. 3: FIG. 3 shows a three-dimensional view of the station shown in FIG. 1, but with the handle in the extended position;

FIG. 4: FIG. 4 shows a detailed view of the handle;

FIG. 5: FIG. 5 shows a schematic view of the measuring station and its environment;

FIG. 6: FIG. 6 shows a three-dimensional view of an isolated measuring plate;

FIG. 7: FIG. 7 shows a view from below the measuring plate shown in FIG. 6;

FIG. 8: FIG. 8 shows a schematic view of the components of the measuring station for performing an ESC;

FIG. 9: FIG. 9 shows an electrode arrangement with two independent electrodes per left or right set of electrodes;

FIG. 10: FIG. 10 shows an electrode arrangement with three independent electrodes per left or right set of electrodes;

FIG. 11: FIG. 11 shows an electrode arrangement with four independent electrodes per left or right set of electrodes;

FIG. 12: FIG. 12 shows an electrode arrangement with five independent electrodes per left or right set of electrodes;

FIG. 13: FIG. 13 shows an electrode arrangement with six independent electrodes per left or right set of electrodes;

FIG. 14: FIG. 14 shows an electrode arrangement with seven independent electrodes per left or right set of electrodes;

FIG. 15: FIG. 15 shows an electrode arrangement with larger end electrodes;

FIG. 16: FIG. 16 shows a schematic diagram of one part of the switch;

FIG. 17: 16 shows a schematic diagram of part of the switch, particularly in the handle;

FIG. 18: FIG. 18 illustrates a number of alternative configurations that the measuring station can take, in particular using the switch shown in FIG. 16 or 17;

FIG. 19: FIG. 19 illustrates a configuration selection process.

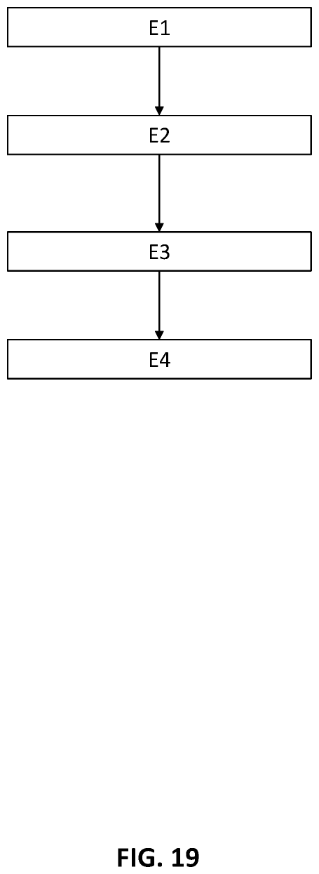

### DETAILED DESCRIPTION

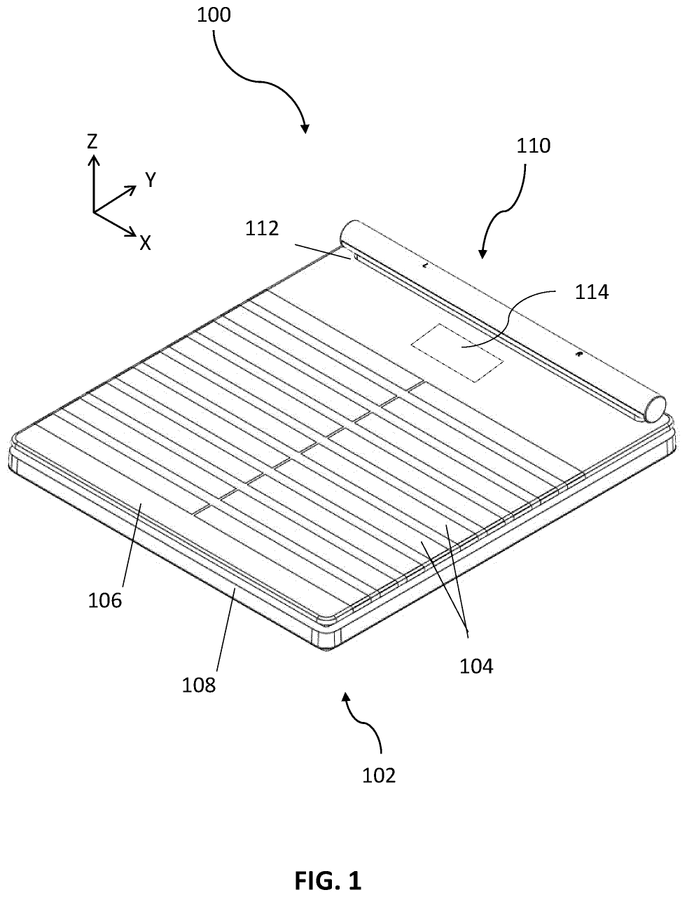

FIGS. 1 to 4 illustrate a measuring station 100 according to at least an embodiment of the present description. The measuring station 100 is mainly in the form of a base 102 on which a user can place his feet, for example flat. The user can stand on the measuring station or sit on a chair. In the normal position of use, the user's feet rest flat on the measuring station 100. The thickness of the base 102 is, for example, less than 10 cm, or even 6 cm. Measuring station 100 comprises one or more sensors 104 capable of measuring physiological information from a user.

In an embodiment, some sensors 104 (e.g. electrodes) are mounted on a substrate 106 of the base 102, the substrate being configured to receive a user's feet. The substrate may be a rigid plate, as shown in the figures, and referred to as a measuring plate 106. The measuring plate 106 defines a plane parallel to an XY plane. The measuring plate 106 may be made of glass. Nevertheless, the substrate may be deformable under the weight of the user. The substrate 106 may be mounted on a base 108, for example rigid, or feet (not shown). In the case of a base 102 functioning as a body scale, sensors are positioned between the substrate 106 and the base 108 (so-called “sandwich” architecture) or between the substrate 106 and the feet (so-called “foot” architecture). The sensors may be load cells (generally four) which can be used to obtain a weight, and therefore a mass, of a user. The base 108 may be made of metal (aluminum, steel, etc.) or plastic.

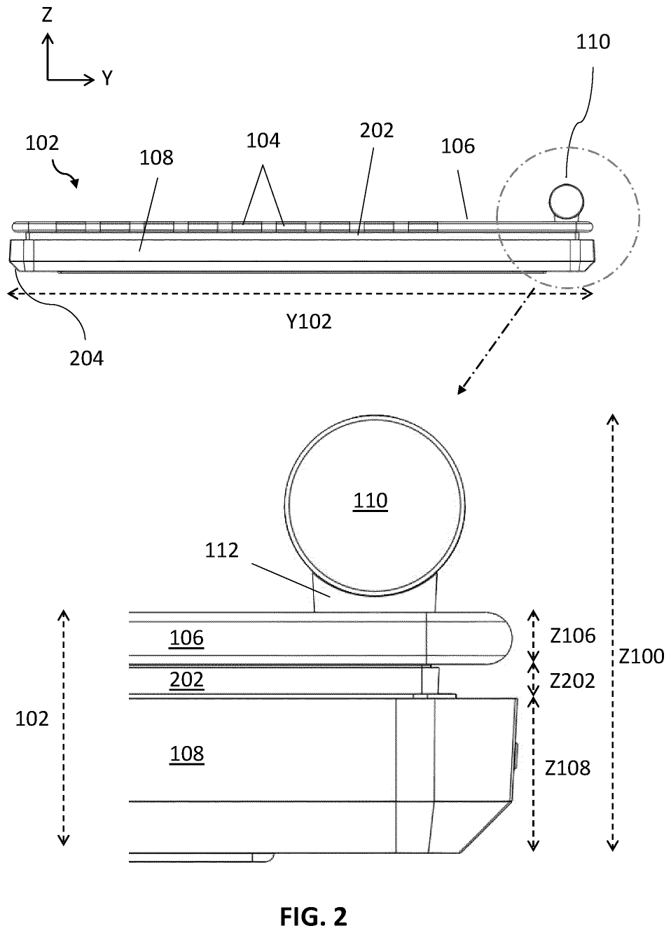

As shown in FIG. 2, the measuring station 100 also comprises a support plate 202, which may be attached to the measuring plate 106. The support plate 202 is designed to accommodate some of the electronics of the measuring station 100, in particular via a printed circuit board (PCB) mounted on the support plate 202. Support plate 202 is therefore positioned between base 108 and measuring plate 106.

In a foot-operated architecture, we define two assemblies that move in relation to each other: the feet on the one hand (fixed assembly), and everything else on the other (moving assembly). Load cells mechanically link these two assemblies. The support plate, if present, is then generally hidden by an external cover integral with the measuring plate. Visually, only the moving part is usually visible.

In sandwich architecture, we define two assemblies that move relative to each other: the base 108 (and associated elements) on the one hand (fixed assembly), and everything else on the other (moving assembly). Load cells mechanically connect these two assemblies. Visually, both assemblies are generally visible.

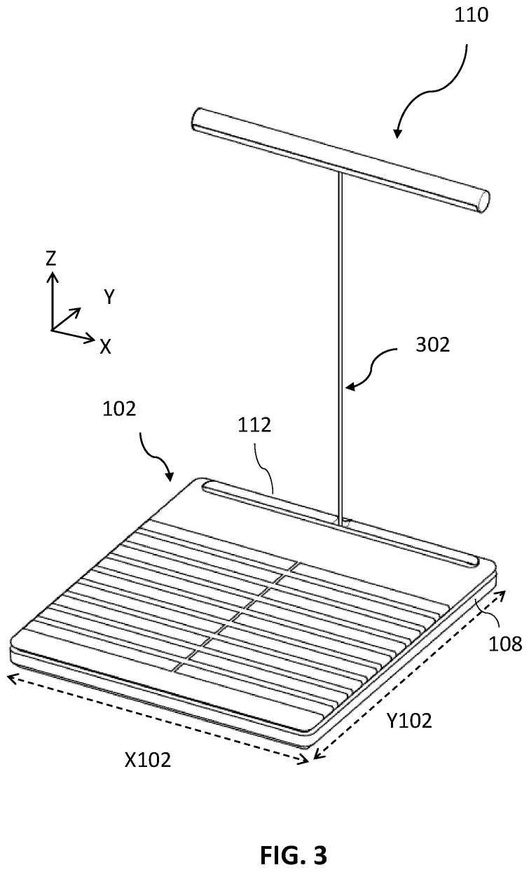

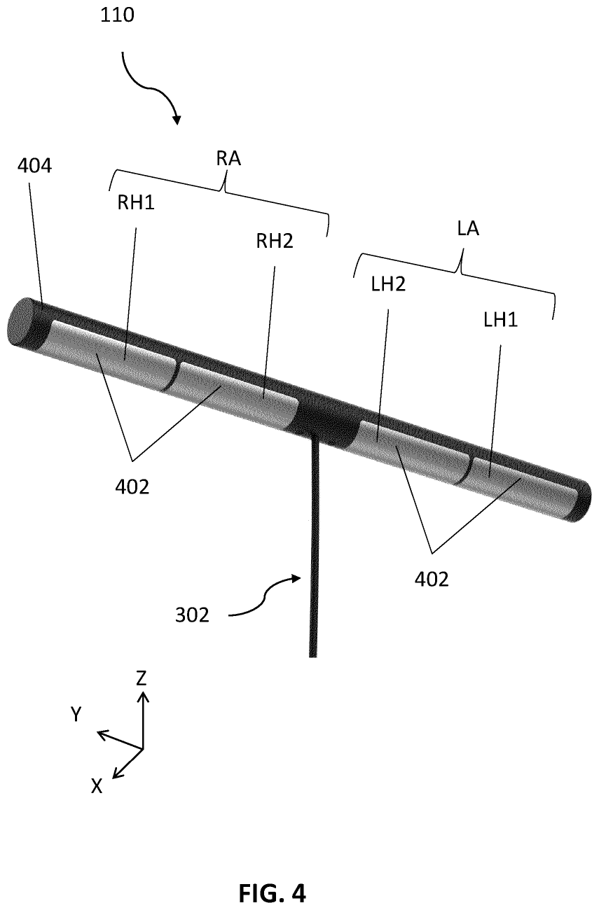

In an embodiment, the measuring station 100 further comprises a handle 110, suitable for being grabbed by at least one hand of the user, illustrated in FIGS. 3 and 4. The handle 110 may be connected to the measuring station by a cable 302 (visible in FIG. 3). In order to provide a convenient measuring station 100 without loose cables, the cable 302 may be extended and retracted (e.g. wound and unwound) inside the base 102. To this end, a reel (not visible in the figures) is arranged in a space between the substrate 106 and the base 108. At least two positions are thus defined for the handle: a stowed position (visible in FIGS. 1 and 2) and a deployed position (visible in FIG. 3). The base 102 further comprises a handle support 112 which can accommodate the handle 110 in the stowed position. Handle support 112 is mounted on substrate 106, for example. It will be described in more detail later. Handle 110 also comprises at least one sensor 402.

Handle 110 is used to perform at least one of the following measurements: ECG (ECG-1-channel between the two hands or several channels with other limbs), BIA (known as “segmental”), IPG, potentially ESC. The sensors on the handle 110 are chosen in particular from: optical sensor for PPG and electrodes.

The base 102 may include a display 114 (e.g. a screen or an LED or e-ink display) for displaying information to the user. The display 114 is shown dotted in FIG. 1 because, in the example of the figures, it is not or only slightly visible when switched off.

The base 108 of the base 102 may include a chamfer 204 to facilitate grabbing of the measuring station 100 when it is on the ground.

In an embodiment, the base 102 has an essentially rectangular shape in an XY plane. For example, base 102 has a substantially parallelepiped shape in XYZ space.

When the measuring station 100 is positioned flat, the measuring plate 106 is parallel to a plane XY. The measuring station 100 comprises a longitudinal direction in a plane XY and a transverse dimension in a plane XY and orthogonal to the longitudinal direction. By height, it is meant the dimension along the Z axis (also known as thickness); by width, it is meant the transverse dimension along the X axis; by length, it is meant the longitudinal dimension along the Y axis. In normal use, the user's feet are positioned along the length Y of base 102. The edge of measuring station 100 (or base 102, or measuring plate 106) that is closest to the front part of the foot in normal use (i.e. the toes) is referred to as the front edge, and the opposite edge of measuring station 100 (or base 102, or measuring plate 106) that is closest to the rear part of the foot in normal use (i.e. the heel) is referred to as the rear edge. A median axis can be defined, along the length Y (i.e. longitudinally), around which the measuring plate 106 is symmetrical and which makes it possible to define a left part, intended for the left foot, and a right part, intended for the right foot. The width X102 of the base 102 may be between 330 and 400 mm (e.g. approx. 357 mm) and the length of the base Y102 may be between 300 and 360 mm (e.g. approx. 325 mm). The length and/or width of the measuring plate 106 may be slightly less than that of the base 108, so that the measuring plate 106 is slightly set back from the base 108. In this case, the length and width of base 108 correspond respectively to the length and width given above for base 102. Such a design protects the measuring plate 106 from impact and contact with the external environment. Application FR2106653, incorporated by reference, describes such a solution.

Specifically, as illustrated in FIG. 2: the height H102 of the base 102 may be between 20 and 35 mm (for example between 35 and 40 mm) and the maximum height H100 of the measuring station 100 may be between 45 and 55 mm (for example 51 mm). As illustrated in the figures, only the handle support 112 and the handle 110 protrude in the Z direction from the measuring plate 106. In detail: the height Z108 of the base 108 may be between 10 and 20 mm (e.g. 18 mm), the height Z202 of the support plate 202 may be between 3 and 6 mm (e.g. 4 mm), the height Z106 of the measuring plate 106 may be between 4 and 8 mm (e.g. 6 mm).

The cable 302 may be between 50 cm and 120 cm long. The length is chosen so that most users can grab the handle while standing with their hands down (at rest).

The measuring station 100 could, however, have different shapes and/or dimensions, provided that the shape and/or dimensions enable the measurements described herein to be obtained. In particular, the base 102 could have an oval or more rounded shape in the XY plane.

The measuring station 100 can have a mass of between 3 and 6 kg (for example, between 4 and 5 kg).

Thanks to the sensor(s) on the base 102 and/or the sensor(s) on the handle 110, the measuring station 100 can perform a set of measurements on the user. In particular, the sensors 104 used comprise electrodes that are formed from electrically conductive paths mounted on the substrate 106 and/or the handle 110 (metal inserts, metal deposits, etc.). Some measurements may require only base 102 sensors, other measurements may require only handle 110 sensors, other measurements may require both handle 110 and base 102 sensors simultaneously.

The measuring station 100 can thus perform an ECG using the handle 110 (e.g. a 1-channel ECG), or an ECG using the handle 110 and base 102 (e.g. a multi-channel ECG, such as a six-channel ECG). Measuring station 100 can thus perform a BIA body impedance measurement analysis using handle 110 and/or base (inter-leg BIA and/or segmental BIA). In this way, the measuring station can perform an IPG in the leg arch (“between legs”) or IPG in the foot (“in the foot”).

The sensors can comprise electrodes capable of: measuring and/or applying a voltage (direct or alternating) and/or a potential (direct or alternating), and/or injecting and/or retrieving a current (direct or alternating). The functions of these electrodes may be chosen from the following list: i+ and i−, for injecting alternating current into the body of a user; V+ and V− for measuring a potential difference in the body of a user; RA, LA and LL, for measuring an electric current passing through the body of a user; sudo_cath, sudo_an, for measuring skin conductivity and sudo_HiZ for fixing the body of a user to a given impedance. Electrodes i+ and i−, V+ and V− are used for BIA, IPG or ICG; electrodes RA, LL, LL are used for ECG; electrodes sudo_cath, sudo_an and sudo_HiZ are used for ESC.

In particular, the measuring station 100 is configured to perform different measurements. As the number of electrodes is limited (due to surface and number considerations), the measuring station 100 features a particular electrode arrangement, with a switch.

As previously mentioned, the sensors may include load cells, which enable the measuring station 100 to measure a weight and perform a BCG.

The handle 110 is illustrated in detail in FIG. 4. The handle 110 enables the measuring station 100 to perform a wider variety of measurements, or even more comprehensive measurements, thanks to an electrical connection with at least one hand, or even both hands. In particular, segmental BIA and/or multi-channel ECG are made possible by the addition of handle 110 to base 102. The sensors 402 on handle 110 comprise, for example, electrodes capable of measuring and/or applying a voltage and/or a potential and/or injecting and/or retrieving a current.

In an embodiment, the handle 110 comprises four electrodes, arranged in two pairs: one pair for the left hand and one pair for the right hand. For this purpose, the electrodes on the handle are referred to as: electrodes LH1, LH2, side by side on a left part of handle 110 and electrodes RH1, RH2, side by side on a right part of the handle (by left part, respectively right part, is meant the part of the handle intended to be in contact with the left hand, respectively right hand). “Side by side” here means with a gap between the electrodes, to insulate the electrodes from each other. The electrodes are therefore arranged one after the other between two ends of handle 110. When the handle 110 is straight, the electrodes are arranged one after the other along the main direction of the handle 110. Electrodes LH1 and RH1 are positioned axially towards one end of the handle; electrodes LH2, RH2 are positioned axially towards the center of the handle. Thus, in order, we have the following electrodes: LH1, LH2, RH2, RH1.

In an embodiment, the sensors 402 of the handle 110, when they are electrodes, are made in the form of several metal inserts in the handle 110. Materials that may be used for metal inserts include stainless steel, titanium, brass, ITO (indium tin oxide), nickel (or nickel alloy), or conductive plastics. For signal processing and/or acquisition, in particular ECG, the handle 110 may include processing electronics (amplifier, follower operation, etc.), in particular for ECG and/or impedance measurement. It is generally preferable to amplify the signal as close as possible to the electrodes, as the cable may pick up ambient noise.

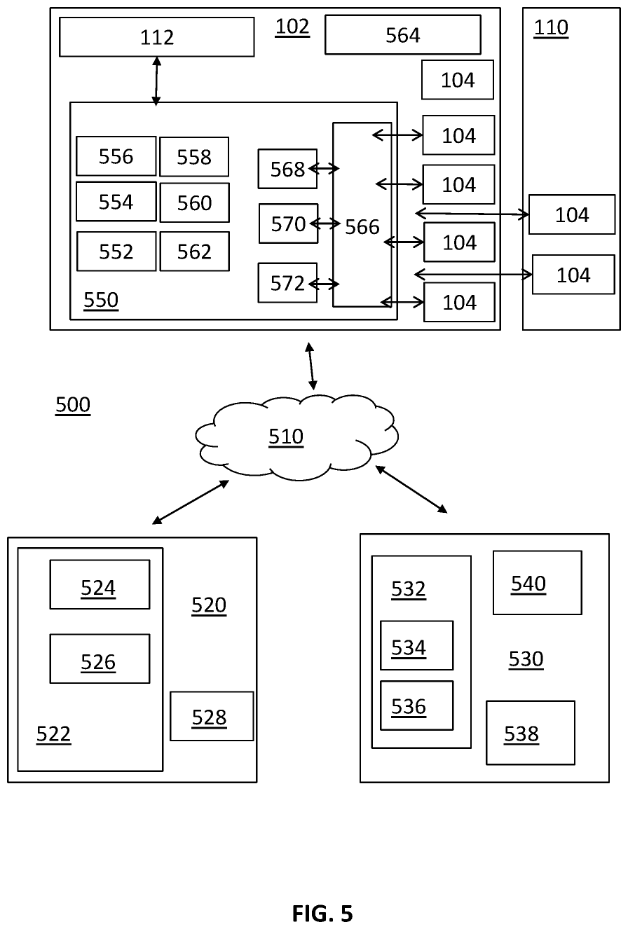

FIG. 5 shows a schematic view of the overall architecture 500 into which the measuring station 100 can be inserted. This overall architecture forms a system comprising the measuring station 100. In particular, the measuring station 100 can communicate with third-party devices via a communication network 510, which is for example a wireless network (in particular a network compatible with at least one of the following communication protocols: Bluetooth, Wi-Fi, Ethernet, etc.). The third-party devices may comprise a server 520 and a mobile terminal 530 (smartphone, etc.). The server 520 may comprise control circuitry 522, including a processor 524 and memory 526, and an input/output (I/O) interface 528, which enables the control circuitry to receive and send data to the communication network 510. The mobile terminal 530 may comprise control circuitry 532, including a processor 234 and memory 236, and an input/output (I/O) interface 538, which enables the control circuitry to receive and send data. Server 520 is a remote server, for example located in a data center. The mobile terminal 530 further comprises a user interface 540 (“UI”) configured to display information to the user and enable him/her, where appropriate, to enter information (such as height, gender, etc.). In particular, control circuitry 532 is configured to run an application managing the environment of measuring station 100. The mobile terminal 530 is a personal object of the user, generally close to the user.

The measuring station 100 can communicate with the server 520 and/or the mobile terminal 530. In an embodiment, the measuring station 100 can communicate directly with the mobile terminal 530, for example via Bluetooth or Bluetooth Low Emission (BLE). This communication may be implemented when the measuring device 100 is installed, in particular to pair it with the 530 mobile terminal and/or to configure a connection to the server 520 that does not transit via the 530 mobile terminal and/or as a backup to a faulty communication with the server 520. In an embodiment, the measuring station 100 can communicate directly with the server 520, without passing through the mobile terminal 530. This communication enables the user to use the measuring station even without having his mobile terminal 530 nearby.

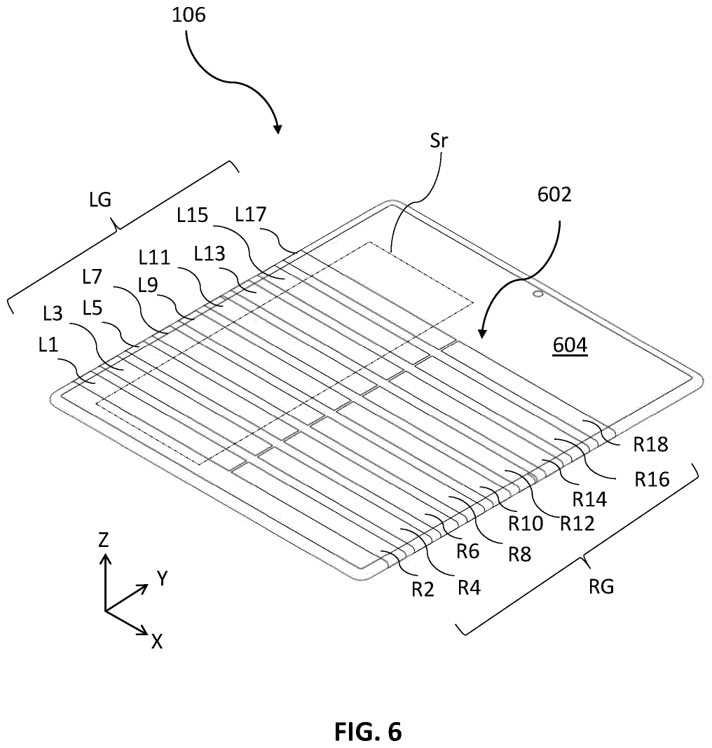

The measuring station 100 also comprises control circuitry 550 with a processor 552 and a memory 554, and an input/output (I/O) interface 556, which in particular enables the control circuitry to receive and send data to the communications network 510. Processor 552 is configured in particular to process data obtained by sensors 104. In particular, processor 552 can execute instructions from a program stored in memory 554. Control circuitry 550 may comprise a microcontroller, which integrates processor 552, memory 554 and input/output interface 556. Control circuitry 550 may also include an analog front end (AFE). Control circuitry 550 may also include an analog-to-digital converter (ADC). The measuring station 100 comprises a voltage source (e.g. direct) 558 and a current source 560 (e.g. alternating). The measuring station 100 also comprises a voltmeter 562 (or any system for measuring voltage). Voltmeter 562 may be integrated into the AFE. The current source 560 may be integrated into the AFE, and the voltage source 558 may be integrated into the MCU microcontroller (e.g. via a digital-to-analog converter DAC). Some sensors 104 (in particular the sensors 402 on the handle 110 in FIG. 4 or the electrodes 602 on the base 102 in FIG. 6) are connected to the control circuitry 550 (for example to the MCU or the AFE). The measuring station 100 comprises a battery 564, suitable for supplying power to the various components of the measuring station 100.

Control circuitry 550 and other electronic components may be mounted on a printed circuit board (PCB), for example attached to support plate 202. Connectors link the measuring plate's electrical conductor paths to the PCB. In order to be able to modify the connections of the electrodes to the various components of the measuring station 100, the measuring station comprises a switch 566. Switch 566, which may comprise a plurality of MCU-controlled switches, will be described in more detail later).

Control circuitry 550 comprises, for example, an ECG acquisition system, an impedance measurement system (for BIA or IPG), an ESC system (for ESC). For each of these systems, various components of the measuring station 100 are used. For example, the ECG acquisition system comprises electrodes (represented by 602 in FIGS. 6 and 402 in FIG. 4) and an ECG 568 electrical circuit (which notably integrates various amplification and/or filtering stages and a demodulator); the impedance measurement system comprises electrodes (represented by 602 on FIGS. 6 and 402 on FIG. 4), the current source 560, the voltmeter 562 and an impedance measurement electrical circuit 570 which connects the electrodes to the current source and to the voltmeter (which incorporates various amplification and/or filtering stages); the ESC system comprises electrodes, the voltage source 558 and an ESC electrical circuit 572 (which incorporates various electronic components, including resistors). Switch 566 is used to connect the electrodes to the various circuits 568, 570, 572 mentioned above, or to disconnect all the electrodes from control circuitry 550.

The control circuitry 550 is essentially located in the base 102, with the exception of a few components (amplification, filtering and switches) located in a PCB in the handle 110, to process the signals before passing them through the cable 302.

As previously mentioned, the measuring station 100 also comprises a display 114, such as a screen (OLED/PMOLED, Retina, etc.), to display information to the user. Alternatively, the measuring station 100 comprises no display.

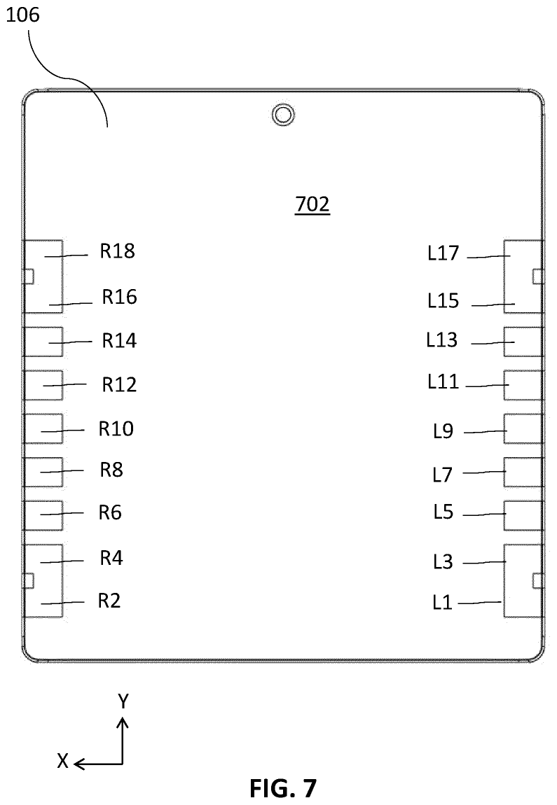

In an embodiment, the sensors 104 include electrically conductive paths 602 (referred to as “electrodes”) on the base 102 (see in particular FIGS. 6 and 7). The electrodes 602 may take the form of a metal deposit on an upper face 604 of the measuring plate 106. The upper face 604 of the measuring plate 106 is defined as the face receiving the user's feet (the visible outer face). For electrical connection to the PCB, the electrodes 602 pass through an edge of the measuring plate 106 and extend to a lower face 702 of the measuring plate 106. The edge (or edges) of the measuring plate 106 may be rounded to ensure proper metal deposition and electrical continuity. In addition, a rounded edge avoids the risk of injury when grabbing the measuring station 100. By rounded edge is meant a circular arc or similar shape. The rounded edge also simplifies metal deposition during manufacturing. Application FR2106653, incorporated by reference, describes these electrically conductive paths in detail.

The electrodes 602 are connected to the PCB via a connector, which makes the connection between the electrical conduction path on the lower face 702 and the PCB mounted on the support plate 202. Switch 566 is used to connect and disconnect the electrodes to the various systems (ECG acquisition system, impedance measurement system, ESC system, etc.). In this way, each electrode can have several different functions, depending on the switching position of switch 566. Switch 556 comprises, for example, a plurality of switches controlled by the MCU.

The upper face 604 of the base 102 comprises a left set LG of electrodes designed to be in contact with the left foot, and a right set RG of electrodes designed to be in contact with the right foot. When the base 102 is installed in normal use, the user places his feet on the left side of the scale and the right side of the scale (with the toes facing the display 114). FIGS. 6 and 7 show electrically conductive paths L1, L3, L5, L7, L9, 11, L13, L15, L17, which form the electrodes of the left set LG of electrodes, and electrically conductive paths R2, R4, R6, R8, R10, R12, R14, R16, R18, which form the electrodes of the right set RG of electrodes.

Electrodes 602 can take the form of strips parallel to each other along direction X (the strips extend along the width X of base 102).

In the illustrated architecture, the pairs of electrically conductive paths L1 and L3; L15 and L17; R2 and R4; R16 and R18 are not independent but are permanently electrically connected, so that the base 102 comprises in practice seven independent electrodes in the left set LG and seven independent electrodes in the right set RG. These permanent electrical connections may be made via the electrically conductive paths on the measuring plate 106 (for example on the lower face 702, not shown) or via the PCB of the measuring station 100.

In one example, the electrically conductive paths on the upper face 604 corresponding to the electrodes 1301-1312 have a dimension (on the upper face 604) along the length Y of between 1.5 cm and 2 cm (e.g. 1.7 cm); the spacing between two strips may be between 0.5 cm and 1 cm (e.g. 0.85 cm); the electrodes can have a dimension along the width X of more than 10 cm.

In particular, each set LG, RG can comprise at least four independent electrodes to perform an IPG in the foot (two electrodes connected to the alternating current source 560 and two electrodes connected to the voltmeter 562). In another embodiment, each set LR, RG may comprise at least two independent electrodes (to perform an ESC with anode/cathode and a high-impedance electrode, or to perform a BIA or IPG between the legs), or three independent electrodes.

In an embodiment, the measuring station 100 comprises a plurality of conductive surfaces (the electrically conductive paths described above) for measuring the conductance of the skin (ESC measurement, in particular for performing an ESC), in particular that of the skin under the feet. In FIG. 6, these conductive surfaces take the form of parallel strips L1 to L17 and R2 to R18. In particular, the base 102 comprises two sets of electrically conductive paths (also known as electrodes): a first set LG of electrodes, which is positioned on a left part of the base 102 in normal use (referred to as the left set, but this term is not to be interpreted restrictively for a left foot only), and a second set RG of electrodes, which is positioned on a right part of the base 102 in normal use (referred to as the right set, but this term is not to be interpreted restrictively for a left foot only). In particular, a user could mount the base 102 upside down (left foot on the right set RG and right foot on the left set LG). FIGS. 1 and 6 illustrate an example of an embodiment of the right and left sets LG, RG. The two sets of electrodes LG, RG are spaced apart by a distance of, for example, between 0.1 cm and 0.5 cm.

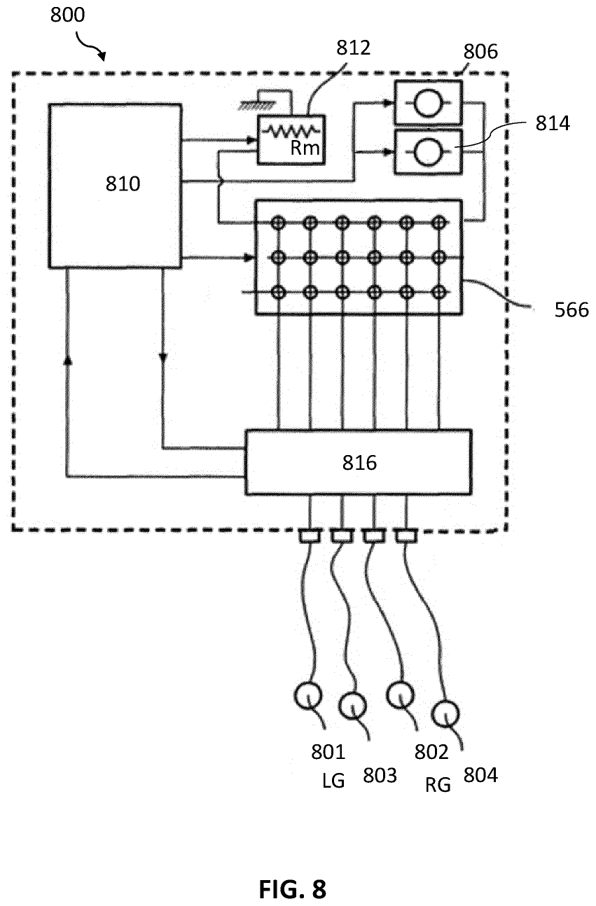

The left LG and right LD sets of electrodes are formed by electrodes mounted on the measuring plate 106. The simplified diagram 800 in FIG. 8 shows four electrodes: electrodes 801 and 803 for the left set LG and electrodes 802 and 804 for the right set RG. The sets RG, LG can include more electrodes, as described below.

Unless explicitly mention of the handle, all the features specific to sweat activity measurement may be carried out with the measuring station 100 comprising only the base 102. It will therefore be referred to the measuring station 100 or the base 102 indifferently.

Left and right sets of electrodes LG, RG enable ESC, i.e. measurement of foot sweat activity. Documents WO2006/136598, WO2008/107324, WO2013/075963, WO2014/033105, WO2015/036530, WO2016/083432 describe a system for performing ESC. The reader is referred to these documents for theory and physiological principles.

The measuring station 100 (the base 102) comprises an ESC system, adapted to perform a measurement of the electrochemical conductance of the skin to assess a sweat function thereof. In particular, the ESC system comprises a direct excitation source 806 (e.g. voltage source 528 which generates a direct voltage or a direct voltage generator), adapted to generate direct voltage signals and in particular constant voltage steps. Each voltage step can last between 0.2 s and 5 s. The voltage delivered by the direct voltage source is, for example, between 0 and 10 V, or even between 0 and 4 V.

The direct voltage source 806 is connected to electrodes 801 to 804, via switch 566, so that the electrodes may be connected or disconnected to the direct voltage source 806. In the case of an ESC, electrodes 801 to 804 are connected in pairs to direct voltage source 806 to operate as anode and cathode respectively. In particular, one electrode from each set LG, RG is connected to the terminals (or pole, for a source) of source 806 to form an anode and cathode pair (for example electrode 801 as anode and electrode 802 as cathode). Two electrodes of the same set LG, RG may be connected to the same pole of source 802 (for example, electrodes 801, 803 as anode and electrodes 801, 804 as cathode).

In the present description, it is possible to reverse the polarities, for example with switch 566 or via voltage generator 806 itself, so that anode and cathode are reversible. Therefore, when it is said that a first electrode is connected to the cathode and a second electrode is connected to the anode, it is also indicated that the anode and cathode may be reversed.

The measuring station 100 further comprises a control unit 810 (e.g. the MCU), able to control the direct voltage source 806 and to control a measuring circuit 812 of an ESC circuit 572. In particular, the measuring circuit 812 enables the control unit 810 to measure the current flowing through the user's feet. To this end, control unit 810 can measure a voltage across a resistor called the measurement resistor Rm. The measuring resistor Rm may be variable and controllable. Control unit 810 may be part of control circuitry 550.

Switch 566 selects two electrodes as anode and cathode, the first being connected to direct voltage source 806 and the second to measuring resistor Rm.

The left set RG or the right set RD comprises at least two electrodes 801, 803 and 802 and 804 which are electrically independent of each other. By electrically independent of each other, it is meant that the two electrodes 801, 803 or 802, 804 are not permanently electrically connected and that it is therefore possible to have a configuration in which, as said two electrodes are electrically independent of each other, the switch 566 can assign them two different functions. Conversely, in an embodiment, switch 566 can electrically connect the two electrodes 801, 803 or 802, 804, so that they have the same function.

Thanks to this independence, the measuring station 100 can perform measurements with an electrode 801 to 804 (from the same LG, RG set) which can take on, at different times, at least two different functions, depending on the configurations enabled by switch 566. For example, measuring station 100 can perform sequentially (one after the other), thanks to switch 566, at least two measurements from among: ESC, BIA, IPG, ICG, ECG (with or without handle 110).

In an embodiment, the left set LG and the set each comprise at least two electrically independent electrodes 801, 803, 802, 804. In this way, each RG and RD set can have several functions: in particular, any measurement that requires two electrodes with different functions in the two sets (IPG between the legs, BIA between the legs, IPG in the foot, etc.).

Several examples of such a configuration will be given below.

In a so-called direct voltage, DC, configuration, at least one of the electrodes 801, 803 of the right set RG and/or at least one of the electrodes 802, 804 of the left set LG is connected to the direct voltage source 806 (the “active” electrode) via switch 566.

In an embodiment, in DC configuration, at least one electrode of each set LG, RG is not connected to the direct voltage source 806 via switch 566. This at least one other electrode of said set may be connected in high impedance (so-called “passive” electrode) by the switch. The high impedance is a value between 500 kOhms and 20 MOhms (for example 10 MOhms). The purpose of the high-impedance passive electrode is to enable control unit 810 to measure the potential of a user's body without creating a current leakage to it. For example, switch 566 connects the electrode to a circuit comprising a resistor of the aforementioned value (with, for example, an operational amplifier).

In another embodiment, in DC configuration, all the electrodes of the LG or RG set are connected to the direct voltage source 806 via switch 566. This ensures maximum electrode surface area at the anode and cathode. In particular, in this embodiment, all electrodes of the left LG set are connected to one terminal of the direct voltage source 806 and all electrodes of the right LG set are connected to the other terminal of the direct voltage source 806. In order to still be able to measure body impedance, handle 110 may be connected in high-impedance mode via switch 566 (not shown in the figures). The user then holds the handle when performing an ESC.

Several surfaces are defined on the upper face 604 of the measuring plate 106: the surface Sa of electrodes which are active electrodes in DC configuration (cumulative surface of all these electrodes), the surface St of all electrodes which are used in DC configuration (active electrodes and passive electrodes). An effective surface area is also defined, which is the surface area of the electrodes that is actually in contact with the foot. To free the definitions from the notion of the user's foot, we define an enlarged effective surface Sr, which may correspond to a virtual rectangle of 30 cm along length Y and 15 cm along width (this is a rectangle within which a foot may be placed). A surface Se of the convex envelope that encompasses the electrodes of the same set LG, RG is also defined. This is the smallest convex surface that comprises all the electrodes of a single LG, RG set.

For a set LG, RG, the surface area of the electrode(s) connected to the direct voltage source 806 in DC configuration represents more than 50% of the surface area of the electrodes of the set LG, RG, or even more than 75%, more than 80%, more than 85%, or even more than 90% (Sa/St ratio). This surface condition will be referred to as the “surface criterion” in the rest of the description. This makes it possible to maximize the total surface area of active electrodes, which activate the sweat function of the feet, in order to improve measurement quality. In addition, the surface area of the electrode(s) connected to the direct voltage source 806 in DC configuration represents more than 50% of the surface area of the convex envelope Se, or even more than 55%, more than 60%, or even more than 80% (Sa/Se ratio). This ensures that the active electrodes in DC configuration are distributed under the foot and not localized in a single spot. Indeed, the distribution of sweat glands can vary between individuals, and it is important to be able to limit the effects of this variation. Similarly, this condition limits the effects of foot placement on base 102. To ensure that the surface area of the electrodes is large enough to cover a significant amplitude of the foot, the distance Dmax between the two end electrodes (including the electrodes themselves) along the length Y may be at least 20 cm, or even 25 cm. Similarly, each electrode extends along the width X for at least 10 cm, or even 15 cm. Alternatively or additionally, the Se/Sr ratio may preferably be greater than 75%, or even 90%, or even greater than 100%.

To integrate the ESC function into the base 102 (i.e. without a handle or electrodes in contact with any region other than the feet), one electrode on the base 102 is configured as a passive, high-impedance electrode. This connection may be permanent (e.g. via a connection on the PCB) or may be activated and deactivated via switch 566.

In practice, there are at least two DC configurations for any pair of electrodes: a DC configuration with anode/cathode and a DC configuration with cathode/anode.

Electrode surface may be calculated in several ways. Ideally, only the effective surface of the electrodes is taken into account, i.e. the surface of the electrodes that is actually in contact with the foot. An enlarged effective surface area may be defined, corresponding to an area of the upper face of the base 102 where the foot is likely to be placed during normal use of the measuring station 100. Finally, a total surface area may be defined, corresponding to the total surface area of the electrodes on the upper face 604. In the case of the strips illustrated in FIG. 6, which all have an identical shape, these three definitions are identical. However, it is possible to envisage configurations in which the effective surface area is unrelated to the total surface area, but the enlarged effective surface area verifies the aforementioned criteria. Within this rectangle, the surface criterion is verified.

In the embodiment illustrated in FIGS. 1 and 6, in DC configuration, eight out of nine bands of a set LG, RG are connected to source 566 and one out of nine bands of the same set LG, RG is connected in high impedance (passive band). The passive electrode band is preferably chosen from L7 or L9 (or R8 or R9 respectively) so as to be at arch level, where the foot's contact with the upper surface 604 is weaker than elsewhere.

The base 102 of the measuring station 100, with or without handle, is configured to perform several measurements, including impedance measurements, such as BIA or IPG. To this end, measuring station 100 comprises an impedance measurement system, capable of performing an impedance measurement of a user's body in response to electrical excitation. In particular, the impedance measurement system may comprise an alternating excitation source 814 (e.g. alternating current source 600 for example or an alternating current generator), adapted to inject an alternating current (e.g. sinusoidal). The current values are known to the person skilled in the art. The alternating current source 814 is connected to the electrodes 801 to 804 by the electronic circuit. Switch 566 is used to connect and disconnect electrodes 801 to 804 to alternating current source 814. In the case of impedance measurement, electrodes 801, 804 are connected in pairs to alternating current source 814, at negative and positive terminals.

In an alternating current, AC, configuration, at least one of the electrodes 801 to 804 (right set RG and/or left set LG) is connected to the alternating current source 814 via switch 566. Several AC configurations are possible: in particular, for a given electrode architecture and given functions, there may be several AC configurations for performing BIA and several AC configurations for performing IPG. Switch 566 is configured to selectively activate or deactivate the AC configuration and/or to alternate between the different AC configurations. The ACf configuration (f for “feet”) is used when two electrodes of the same set LG, RG are connected to the two terminals of the alternating current source 814, as this configuration enables IPG to be performed in the foot; the ACb configuration (b for “between the legs”) when one terminal of the alternating current source 814 is connected to at least one electrode of one set LG, RG and the other terminal of the alternating current source 814 is connected to at least one electrode of the other set RG, LG, as this configuration makes it possible to perform an IPG between the legs or a BIA between the legs.

Unless explicitly stated otherwise, any ACf configuration described for one set of LG, RG electrodes applies to the other set of RG, LG electrodes (symmetry between the two sets). On the other hand, the left foot ACf configuration is not activated at the same time as the right foot ACf configuration (for component-related reasons).

Switch 566 may be configured to selectively switch between at least one of the DC configurations and at least one of the AC configurations.

AC configurations include those in which both terminals of the alternating current source 814 are connected to at least two electrodes from the left LG and right RG sets: these include ACf (IPG in the foot) and ACb (IPG leg-arch or BIA without handle). There are also configurations where only one terminal of the alternating current source 814 is connected to at least one electrode from the left LG and right RG sets; in this case, the measuring station 100 comprises the handle 110 and the other terminal is connected to an electrode of the handle (segmental BIA in particular). This configuration will be referred to as ACs (“s” for segmental, as it enables segmental BIA to be performed).

In AC configuration, control unit 810 may need to measure a potential on a user's body (feet and/or hands). For this purpose, measuring station 100 comprises a voltmeter 562 capable of measuring a potential difference between two electrodes. Switch 566 is used to connect a pair of electrodes (e.g. from electrodes 801 to 804, but also from the handle electrodes) to the terminals of voltmeter 562, in order to measure a potential difference.

In practice, the current source(s) may not have two terminals as shown in the schematic diagrams. Nevertheless, there is a pole i+ or i−, V+ or V−, which are determined by the electrical circuit. The term “terminals” refers to these poles.

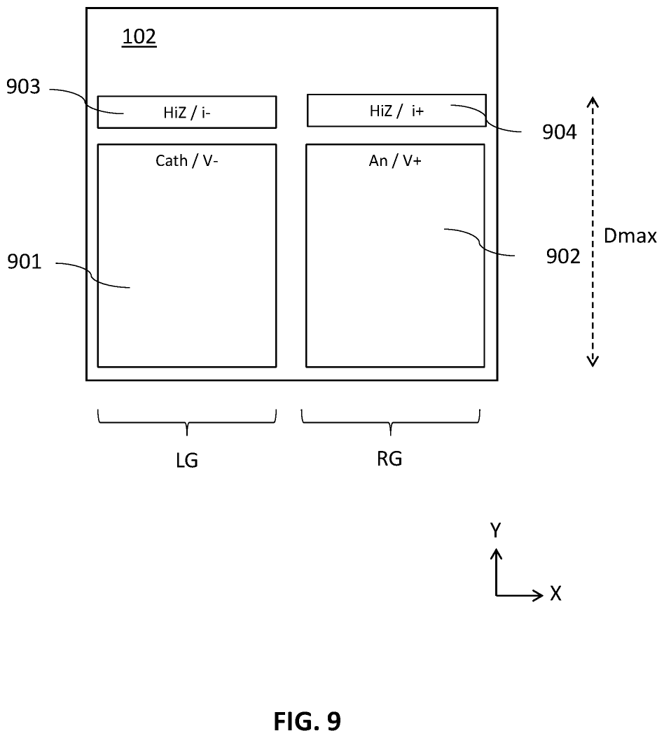

Various arrangements of electrically conductive paths forming electrodes will be presented, in relation to FIGS. 9 to 15. Each arrangement is limited by physical constraints: the arrangement of the electrically conductive paths along the length Y must allow the foot to be in contact with said electrically conductive paths; consequently, the maximum distance Dmax (see FIGS. 9 to 15) between the end electrodes along the length Y in a set LG, RG is limited. The arrangements may be symmetrical about a directional axis Y.

In DC configuration, the pair of electrodes connected to the direct voltage source 806 comprises at least one electrode of the left set LG and at least one electrode of the right set RD. For example, the left set LG contains the cathode and the right set RG contains the anode, or vice versa. On the other hand, two electrodes of the same set LG, RG cannot be connected to both terminals of the direct voltage source 806 (within the same set LG, RG, there cannot be simultaneously one electrode operating as cathode and another electrode operating as anode).

In FIGS. 9 to 15, “HiZ” means that, in a DC configuration, the electrode is connected to the high impedance; “An” or “Cath” means that, in a DC configuration, the electrode is connected to the direct voltage source 806 (either terminal, respectively); i+ or i− means that, in an AC configuration, the electrode is connected to the alternating current source 814 (one or other of the terminals, respectively); V+ or V− means that, in an AC configuration, the electrode is connected to the voltmeter 562 (one or other of the terminals, respectively); “-” means that, whatever the configuration, the electrode is inactivated (e.g. not electrically connected, disconnected by switch 556). The same electrode can therefore have several of these symbols, since it can, depending on the configuration determined by switch 566, sequentially perform several functions.

FIG. 9 illustrates an arrangement 900 in which each LG, RG set comprises two independent electrodes 901, 903 (LG set) and 902, 904 (RG set). The electrodes' functions shown in FIG. 9 are in the form “DC/ACb”.

In DC configuration, switch 566 connects electrodes 901 to direct voltage source 806 (anode/cathode or cathode/anode). In a variant (not shown), electrodes 903 and 904 are also connected to the direct voltage source 806 (electrode 901 with electrode 903 and electrode 902 with electrode 904). In one variant (illustrated), switch 566 connects electrodes 903 and 904 to the high impedance (HiZ). In the example shown, passive electrodes 903 and 904 are located along length Y, below electrodes 901, 902. However, in this configuration, there is a risk that the user's foot will not be in contact with electrodes 903 and 904. The surface area of electrodes 901, 903, 902, 904 is chosen so that the surface area of the electrodes active in the DC configuration satisfies the above-mentioned surface criterion.

In AC configuration, switch 566 connects at least one electrode 901, 902, 903, 904 to alternating current source 814. In particular, in the ACb configuration, switch 566 connects the pair of electrodes 901 and 902 to the terminals of voltmeter 562, and switch 566 connects electrode pair of electrodes 903 and 904 to the terminals of alternating current source 814. Arrangement 900 enables a BIA between the legs or an IPG between the legs.

In the case of an ACs configuration (segmental BIA) with station 100 including handle 110, some of the electrodes of the left set LG or the right set RG may be connected to one terminal of alternating current source 814 or to voltmeter 562 without other electrodes of the same set LG, RG being connected to the other terminal of alternating current source 814 or to voltmeter 562.

The configuration 900 thus enables an ESC, on a base 102, covering most of the foot (with, in particular, separation of the right and left feet made possible by the high-impedance electrode) to be performed, while the base 102 is versatile enough to perform impedance measurement between the legs (BIA, IPG). Switch 566 enables toggling between DC and AC configurations.

However, such an arrangement may have disadvantages, particularly with regard to the positioning of the foot, which may not be in contact with electrodes 903, 904. Nevertheless, enlarging these electrodes means that the surface area of electrodes 901, 902 for the ESC is reduced, which is undesirable. In addition, the number of measurements that may be made is limited (e.g. by ACf configuration, to make an IPG in the foot).

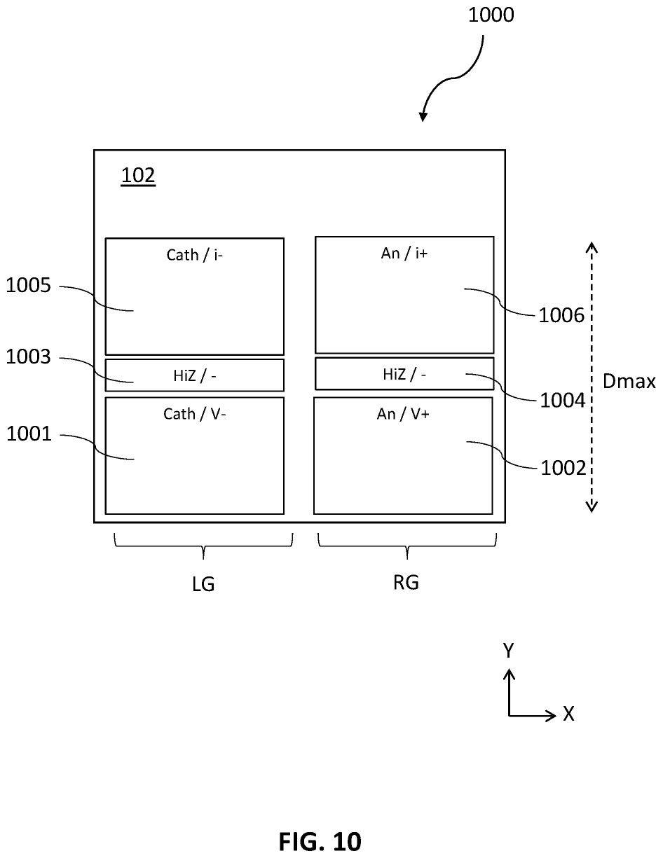

FIG. 10 illustrates an arrangement 1000 without the above-mentioned disadvantages. Each LG, RG set comprises three independent electrodes 1001, 1003, 1005 (LG) and 1002, 1004, 1006 (RG). The electrodes' functions shown in FIG. 10 are in “DC/ACb” form.

In this arrangement, electrode 1003 (respectively 1004) is located along length Y between electrodes 1001, 1005 (respectively 1002, 1006).

In DC configuration, switch 566 connects electrodes 1001, 1005 (LG) and 1002, 1006 (RG) to direct voltage source 806 (anode/cathode or cathode/anode). In a variant not illustrated, electrodes 1003 and 1004 are also connected to the direct voltage source 806 (electrode 1003 with electrodes 1001 and 1005 and electrode 1004 with electrodes 1002 and 1006); in an illustrated variant, switch 566 connects electrodes 1003 and 1004 to the high impedance HiZ.

The surface area of the electrodes is chosen so that the surface area of the active electrodes in DC configuration satisfies the above-mentioned surface area criterion. This means that the surface area of the passive (high-impedance) electrode is smaller than the surface area of an active electrode. In particular, in this arrangement, electrodes 1001, 1005, 1002, 1006 may be large enough for Se/Sr to be greater than 100%. In one example, electrodes 1001, 1002, 1005, 1006 have a dimension along length Y of at least 10 cm (e.g. 14 cm); electrodes 1004 have a dimension along length Y of between 1.5 cm and 2.5 cm; electrodes can have a dimension along width X of more than 10 cm.

In ACb configuration, switch 566 connects electrodes 1001 and 1002 to voltmeter 562, and switch 566 connects electrodes 1005 and 1006 to alternating current source 814. The connection between the negative and positive terminals may be reversed. In ACb configuration, electrodes 1003, 1004 are inactive. Arrangement 1000 enables BIA between the legs or IPG between the legs.

The configuration 1000 thus enables an ESC, on a base 102, covering most of the foot (with separation of the right and left foot possible thanks to the high-impedance electrode) to be performed, while at the same time having a versatile base 102 that can perform impedance measurement. Thanks to the positioning of electrodes 1003, 1004 in the middle of the foot, and to the possibility of having large electrodes 1001, 1005, sensitivity to the position of the foot along length Y is fairly low, which ensures good repeatability of measurements.

Such an arrangement is particularly suitable when no ACf configuration is required, i.e. no IPG in the foot.

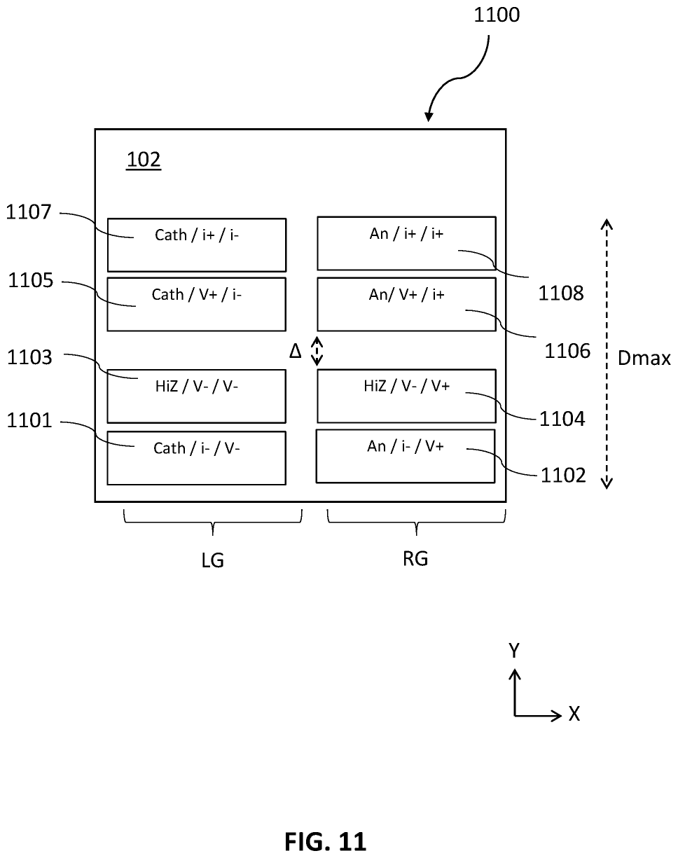

FIG. 11 illustrates an arrangement 1000 without the above-mentioned disadvantages. Each LG, RG set comprises four independent electrodes 1101, 1103, 1105, 1107 and 1102, 1104, 1106, 1108. The electrodes' functions shown in FIG. 11 are in the form “DC/ACf (right and left)/ACb”. Right and left ACf arrangements are not implemented simultaneously (for reasons of data acquisition by the control circuitry and switch).

In DC configuration, switch 566 connects electrodes 1101, 1105, 1107 (LG) and 1102, 1106, 1108 (RG) to the direct voltage source (anode/cathode or cathode/anode). In a variant not shown, electrodes 1103 and 1104 are also connected to the direct voltage source 806 (connected to the same terminal as the other electrodes in their set of electrodes); in an illustrated variant, switch 566 connects electrodes 1103 and 1104 to high impedance HiZ. Electrodes 1103 and 1105 (respectively 1104 and 1106) may be reversed.

The surface area of the electrodes is chosen so that the surface area of the active electrodes in the DC configuration satisfies the above-mentioned criterion. In particular, if electrodes 1101 to 1108 all have the same surface area, the ratio of active electrode surface area to total surface area is 75%.

Several AC configurations are possible. In an ACf configuration, switch 566 connects electrodes 1101 and 1107 to the terminals of alternating current source 814, and switch 566 connects electrodes 1103 and 1105 to the terminals of voltmeter 562. This configuration enables an IPG to be made in the foot.

In an ACb configuration, switch 566 connects one or two of electrodes 1101, 1103 and one or two of electrodes 1102, 1104 to the terminals of voltmeter 562 and connects one or two of electrodes 1105 and 1107 and one or two of electrodes 1106 or 1108 to the terminals of alternating current source 814. This ACb configuration makes it possible to perform a leg-arch IPG or BIA between the legs.

In the case of an ACs configuration (segmental BIA) with station 100 including handle 110, some of the electrodes of the left set LG or the right set RG may be connected to one terminal of alternating current source 814 or to voltmeter 562 without other electrodes of the same set LG, RG being connected to the other terminal of alternating current source 814 or to voltmeter 562.

Arrangement 1100 thus enables a segmental BIA, a BIA between the legs, an IPG between the legs and an IPG in the foot.

Arrangement 1100 thus enables an ESC to be performed on a base 102 that covers most of the foot (with, in particular, separation of the right and left feet possible thanks to the high-impedance electrode), while at the same time providing a versatile base 102 that can perform impedance measurement with a high degree of adaptability.

However, in the ACf configuration (for IPG in the foot), arrangement 1100 offers two electrodes 1103 or 1105 (or 1104 and 1106) which are connected to the two terminals of the alternating current source 814 while being fairly close to each other (distance Δ in the figures, which is defined as the minimum distance between electrodes V+ and V− in the ACf configuration). This proximity may result in a loss of signal quality when the voltage is obtained by voltmeter 562. It may then be possible to space electrodes 1103 and 1105 apart (and 1104 and 1106 apart), but then, because of the constraint of distance Dmax, the size of electrodes 1101 to 1108 must decrease. In the DC configuration, this means that the surface area of the anodes or cathodes in contact with the foot decreases, which creates difficulties for ESC measurement.

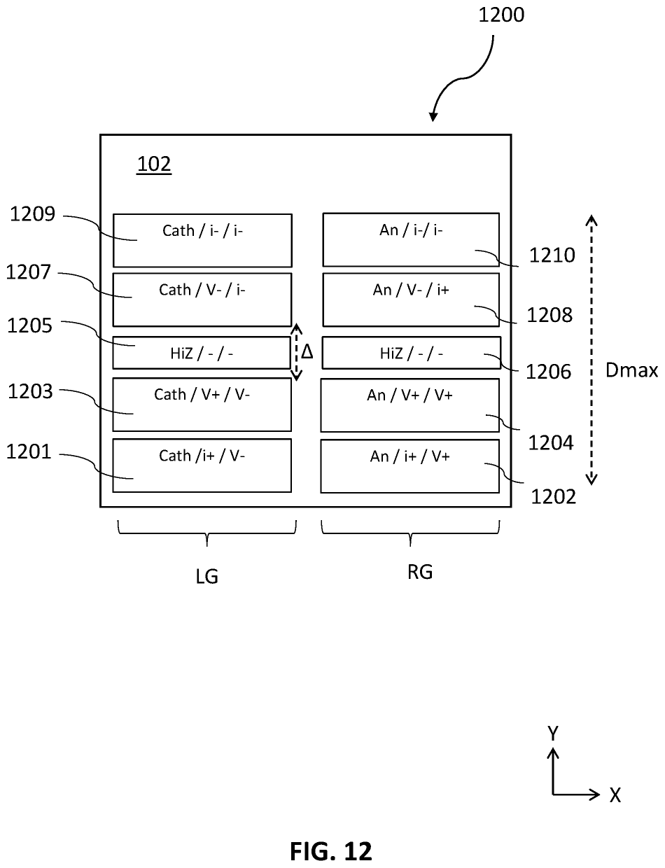

FIG. 12 illustrates an arrangement 1200 which overcomes the difficulty of arrangement 1100. Each LG, RG set comprises five independent electrodes 1201, 1203, 1205, 1207, 1209 and 1202, 1204, 1206, 1208, 1210. The electrodes' functions shown in FIG. 12 are in the form “DC/ACf (right and left)/ACb”. Right and left ACf arrangements are not implemented simultaneously (for reasons of data acquisition by the control circuitry and switch operation).

In DC configuration, switch 566 connects electrodes 1201, 1203, 1207, 1209 and 1202, 1204, 1208, 1210 to direct voltage source 806 (anode/cathode or cathode/anode). In a variant not shown, electrodes 1205 and 1206 are also connected to direct voltage source 806 (connected to the same terminal as the other electrodes in their set of electrodes); in an illustrated variant, switch 566 connects electrodes 1205 and 1206 to high impedance HiZ. Electrode 1205 (respectively 1206) may be reversed with electrode 1203 or 1207 (respectively 1206 with 1204 or 1208).

The surface of the electrodes is chosen so that the surface of the electrodes that are active in the DC configuration verifies the above-mentioned surface criterion. The configuration with five independent electrodes makes it possible to verify the aforementioned surface criterion without compromising the possibility of performing a plurality of different measurements (ESC, BIA, IPG, etc.) or the quality of the measurements. In particular, if electrodes 1201 to 1210 all have the same surface area, the ratio of active electrode surface area to total surface area is 80%. If electrodes 1205, 1206 (electrodes configured to be passive in DC configuration) are smaller (as illustrated), this ratio increases.

As with arrangement 1100, the ACf configuration is possible because each LG, RG set of electrodes comprises at least four independent electrodes. In an ACf configuration, switch 566 connects electrodes 1201 and 1209 (1202 and 1210 for the right ACf arrangement) to the terminals of alternating current source 814, and switch 566 connects electrodes 1203 and 1207 (1204 and 1208 for the right ACf arrangement) to the terminals of voltmeter 562. This configuration enables an IPG to be made in the foot. Switch 566 can deactivate electrodes 1205 (1206 for right ACf arrangement) by disconnecting or grounding them.

In an ACb configuration, switch 566 connects one or two of electrodes 1201 and 1203 and one or two of electrodes 1202 and 1204 to the terminals of voltmeter 562 and connects one or two of electrodes 1207 and 1209 and one or two of electrodes 1208 or 1210 to the terminals of voltage source 814. This ACb configuration makes it possible to perform a leg-arc IPG or BIA between the legs.

In the case of an ACs configuration (segmental BIA) with station 100 including handle 110, some of the electrodes of the left set LG or the right set RG may be connected to one terminal of alternating current source 814 or voltmeter 562 without other electrodes of the same set LG, RG being connected to the other terminal of alternating current source 814 or voltmeter 562.

In this 1200 arrangement, passive electrodes 1205, 1206 may not be activated in ACf configuration (for the IPG in the foot). This means that the distance Δ may be increased compared with arrangement 1100, by placing an inactive electrode between the electrodes connected to the alternating current source in ACf configuration. This improves the quality of impedance measurement signals (IPG in the foot), while having a larger anode and cathode surface area compared with arrangement 1100. The passive electrode (HiZ) may be smaller than the others; as there are four other independent electrodes, ACf and ACb configurations can all be implemented.

Arrangement 1200 enables a BIA between the legs, an IPG between the legs and an IPG in the foot.

In an embodiment of arrangement 1200, electrodes 1205 and 1203 (respectively 1206 and 2014) may be inverted, or electrodes 1205 and 2017 (respectively 1206 and 2018) may be inverted.

Arrangement 1200 thus enables an ESC to be performed on a base 102 covering most of the foot (with separation of the right and left feet possible thanks to the high-impedance electrode), while at the same time providing a highly versatile base 102 that can perform impedance measurement with a high degree of granularity, in particular IPG in the foot with an increased distance Δ between electrodes for good signal quality.

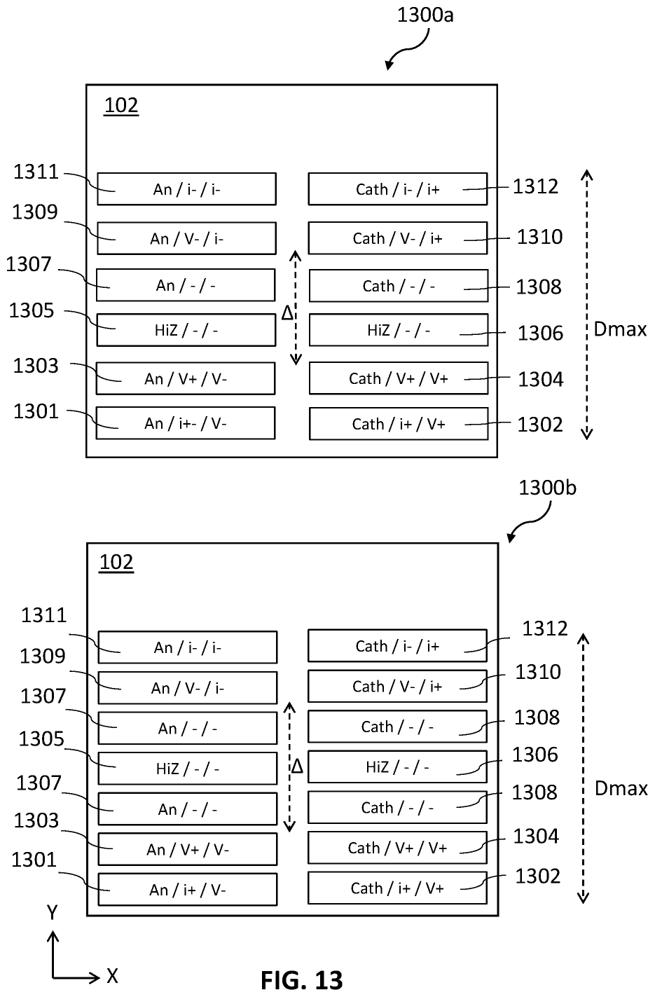

FIG. 13 illustrates two arrangements 1300a, 1300b which further increase the distance Δ between the electrodes while maintaining good quality for the ESC. Each set LG, RG comprises six independent electrodes 1301, 1303, 1305, 1307, 1309, 1311, 1313 and 1302, 1304, 1306, 1308, 1310, 1312. The electrodes' functions shown in FIG. 9 are in the form “DC/ACf (right and left)/ACb”. Right and left ACf arrangements are not implemented simultaneously (for reasons of data acquisition by the control circuitry and switch operation).

In DC configuration, switch 566 connects electrodes 1301, 1303, 1307, 1309, 1311, 1313 (LG) and 1302, 1304, 1308, 1310, 1312 (RG) to direct voltage source 806 (anode/cathode or cathode/anode). In a non-illustrated variant, electrodes 1305 and 1306 are also connected to direct voltage source 806 (connected to the same terminal as the other electrodes in their set of electrodes); in an illustrated variant, switch 566 connects electrodes 1305 and 1306 to high impedance HiZ. Electrodes 1305 and 1307 (1306 and 1308 respectively) may be reversed. Central electrodes along length Y are preferred for passive electrodes during ESC. The surface area of the electrodes is chosen so that the surface area of the active electrodes in the DC configuration satisfies the above-mentioned surface criterion. The configuration with six independent electrodes enables the above-mentioned surface criterion to be verified even more clearly, without compromising the possibility of carrying out a plurality of different measurements (ESC, BIA, IPG, etc.) or the quality of the measurements. In particular, if electrodes 1301 to 2012 all have the same surface area, the ratio of active electrode surface area to total surface area is over 83% (5/6). If electrodes 1305, 1306 (electrodes configured to be passive in DC configuration) are smaller than the others, the ratio is even better.

As with arrangements 1100 and 1200, the ACf configuration is possible because each set of electrodes LG, RG comprises at least four independent electrodes. In an ACf configuration, switch 566 connects electrodes 1301 and 1311 (1302 and 1312 respectively) to the terminals of alternating current source 814, and switch 566 connects electrodes 1303 and 1309 (1304 and 1310) to the terminals of voltmeter 562. This configuration enables an IPG to be made in the foot. Switch 566 can deactivate electrodes 1305, 1306, 1307, 1308 by disconnecting or grounding them.

In an ACb configuration, switch 566 connects one, two or three of electrodes 1301, 1303, 1305 (e.g. two, as shown) and one, two or three of electrodes 1302, 1304, 1306 (e.g. two, as shown) to the terminals of voltmeter 562 and connects one, two or three of electrodes 1307, 1309 and 1311 (e.g. two, as shown) and one, two or three of electrodes 1308, 1310 and 1312 (e.g. two, as shown) to the terminals of voltmeter 562, two or three of the electrodes 1307, 1309 and 1311 (for example two, as illustrated) and one, two or three of the electrodes 1308, 1310 and 1312 (for example two, as illustrated) to the terminals of the alternating current source 814. This ACb configuration makes it possible to perform an leg-arch IPG or BIA between the legs.

In ACs configuration, (segmental BIA) with station 100 including handle 110 some of the electrodes of the left set LG or the right set RG may be connected to alternating current source 814 or voltmeter 562 without other electrodes of the same set being connected to alternating voltage source 814 or voltmeter 562.

In this arrangement 1300a, electrodes 1305, 1307 (and 1306, 1308) may not be activated in ACf configuration (for IPG in the foot). This means that the distance Δ may be increased, compared with arrangement 1200, by placing two inactive electrodes between the electrodes connected to the alternating current source in ACf configuration. This improves the quality of impedance measurement signals (IPG in the foot), while having a higher Sa/ST ratio (that of the surface criterion) compared with arrangement 1200.

The layout 1300a allows for a BIA between the legs, an IPG between the legs and an IPG in the foot.

In a variant of arrangement 1300a, not shown, electrodes 1305 and 1307 (respectively 1306 and 1308) may be reversed.

Arrangement 1300b is a variant of arrangement 1300a in which, for each of the sets RG, LG, electrode 1307 has a larger surface area than the others, in particular electrodes 1303 or 1309. In particular, electrode 1307 extends along length Y on either side of electrode 1305 (thus forming a double electrode). With arrangement 1300b, the distance Δ is further increased by placing three inactive electrically conductive paths (one electrode and one double electrode) between the electrodes connected to the alternating current source in ACf configuration. In this arrangement, electrode 1307 is preferably inactive in AC configuration (ACb in particular). The Se/Sr ratio may be greater than 100% (or at least 90%), thanks to the multiplicity of electrodes along the length Y of base 102. In one example, the strips on the upper face corresponding to electrodes 1301-1312 have a dimension along length Y of between 1.5 cm and 2 cm (e.g. 1.7 cm); the spacing between two successive strips may be between 0.5 cm and 1 cm (e.g. 0.85 cm); the electrodes may have a dimension along width X greater than 10 cm.

Arrangements 1300a, 1300b enable an ESC to be performed on a base 102 covering most of the foot (with separation of the right and left feet possible thanks to the high-impedance electrode), while the highly versatile base 102 can perform impedance measurement with a high degree of granularity, in particular IPG in the foot with a large distance Δ between electrodes for better signal quality.

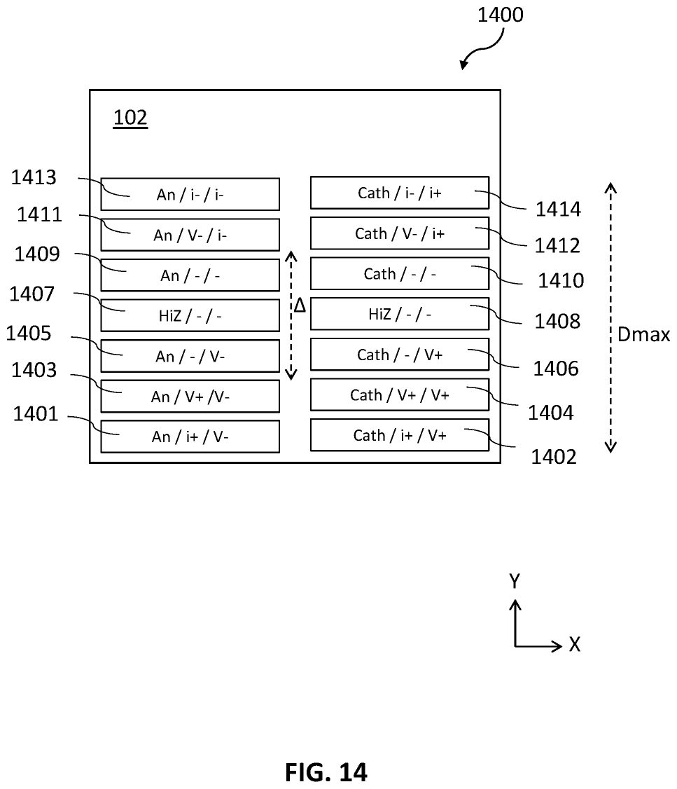

FIG. 14 illustrates an arrangement 1400 which shares a number of the features of arrangement 1300b, except that instead of doubling electrode 1307, a further independent electrode is added. Each set LG, RG thus comprises seven independent electrodes 1401, 1403, 1405, 1407, 1409, 1411, 1413 (LG) and 1402, 1404, 1406, 1408, 1410, 1412, 1414 (RG). The electrodes' functions shown in FIG. 12 are in the form “DC/ACf (right and left)/ACb”. Right and left ACf arrangements are not implemented simultaneously (for reasons of data acquisition by the control circuitry and switch operation).

In DC configuration, switch 566 connects electrodes 1401, 1403, 1405, 1409, 1411, 1413, 1413 (LG) and 1402, 1404, 1406, 1410, 1412, 1414 (RG) to direct voltage source 806 (anode/cathode or cathode/anode). In a variant not shown, electrodes 1407 and 1408 are also connected to direct voltage source 806 (connected to the same terminal as the other electrodes in their LG, RG set of electrodes); in an illustrated variant, switch 566 connects electrodes 1407 and 1408 to high impedance HiZ. Electrode 1407 may be reversed with one of electrodes 1405 or 1409 (1406 and 1408 respectively). Central electrodes are preferred for passive electrodes during ESC. The surface area of the electrodes is chosen so that the surface area of the active electrodes in the DC configuration satisfies the above-mentioned surface criterion. The configuration with seven independent electrodes enables even greater verification of the above-mentioned surface criterion without compromising the possibility of performing a plurality of different measurements (ESC, BIA, IPG, etc.) or the quality of the measurements. In particular, if electrodes 1401 to 1414 all have the same surface area, the ratio of active electrode surface area to total surface area is over 85% (six strips out of seven).

As with arrangements 1100, 1200, 1300a, 1300b, the ACf configuration is possible because each set of electrodes LG, RG comprises at least four independent electrodes. In ACf configuration, switch 566 connects electrodes 1401 and 1413 (respectively 1402 and 1414) to alternating current source 814, and switch 566 connects electrodes 1403 and 1411 (respectively 1404 and 1412) to voltmeter 562 terminals. Switch 566 can deactivate electrodes 1405, 1406, 1407, 1408, 1409, 1410 by disconnecting or grounding them. This configuration enables an IPG to be made in the foot.

In an ACb configuration, switch 566 connects one, two or three of electrodes 1401, 1403, 1405 (e.g. all three, as shown) and one, two or three of electrodes 1402, 1404, 1406 (e.g. all three, as illustrated) to the terminals of voltmeter 562 and connects one, two or three of electrodes 1409, 1411, 1413 (for example two, as illustrated) and one, two or three of electrodes 1410, 1412, 1414 (for example two, as illustrated) to the terminals of alternating current source 814. This ACb configuration makes it possible to perform an arched-leg IPG or a BIA between the legs.

In ACs configuration (segmental BIA) with station 100 which includes handle 110 some of the electrodes of the left set LG or the right set RG may be connected to alternating current source 814 or voltmeter 566 without other electrodes of the same set being connected to alternating current source 814 or voltmeter 566.

In this arrangement 1400, electrodes 1405, 1407, 1409 may not be activated in AC configuration for IPG in the foot. This means that the distance Δ is increased, compared with arrangement 1200, by placing three inactive electrodes between the electrodes connected to the alternating current source in ACf configuration. This improves the quality of impedance measurement signals (IPG in the foot).

Arrangement 1400 enables a BIA between the legs, an IPG between the legs and an IPG in the foot.

In a variant of arrangement 1400, electrodes 1405 and 1407 (2206 and 2208 respectively) may be reversed.

The arrangement 1400 thus enables an ESC to be performed on a base 102 covering most of the foot (with separation of the right and left foot possible thanks to the high-impedance electrode), while at the same time providing a highly versatile base 102 that can perform impedance measurement with a high degree of granularity, in particular IPG in the foot with a distance Δ between electrodes optimized for good signal quality. Sensitivity to foot position in X width and Y length is fairly low, ensuring good measurement repeatability.

In this arrangement, three electrodes are inactive in the ACf configuration, which also means that electrodes for BIA or IPG have a reduced surface area, thus improving signal quality. Indeed, current injection and voltage measurement may be better when they are localized on the foot (by “localized” he means on a reduced surface under the foot). Conversely, these three inactive electrodes in ACf configuration become two active electrodes and one passive electrode in DC configuration, so that the surface area of the electrodes is maximized in this configuration. This seven-independent-electrode configuration offers a large active electrode surface in AC configuration, while enabling multiple types of measurement on the feet or on each foot with good signal quality.

In all the AC configurations shown in the description, the connections to the terminals of alternating current source 814 may be reversed (i+ becomes i− and i− becomes i+); similarly, the connections to the terminals of voltmeter 562 may be reversed (V+ becomes V− and V− becomes V+).

In all the AC configurations shown in the description, the connection between alternating current source 814 and voltmeter 562 may be reversed (V becomes i and i becomes V). For ACf configurations, however, it is preferable to have electrodes V+ and V− inside the segment of a user's body through which the current generated between electrodes i+ and i+ flows. In the opposite case, there is no control of current flow for the segments between the i and V electrodes.

In the arrangements described above, some electrodes can perform at least two, three or four different functions. The division into a plurality of strips along length Y, arranged in parallel along width X, enables increased modularity while maintaining good measurement qualities.

In an embodiment, the measuring station 100 may be used to take an ECG. The ECG may be taken between the legs, between the hands (electrodes RA, LA in FIG. 4) or between the hands (electrodes RA, LA) and the legs. One of the electrodes of the left LG set can then be connected by switch 566 as electrode LL (“left leg”). One of the electrodes of the right set RG can then be connected by switch 566 as electrode RG. Electrodes not connected to direct current source 814 or voltmeter 562 are inactive (i.e. disconnected).

In all the arrangements described, the Y dimension (i.e. along the Y direction, i.e. the length of base 102) of the electrodes that are active in the DC configuration may be equal to or greater than the Y dimension of the electrodes that are passive in the DC configuration. This maximizes the surface area of the foot in contact with the active electrodes. Arrangements 1000 and 1200 illustrate a case where the passive electrode 1003, 1004, 1205, 1206 is smaller along the Y length than the active electrodes 1001, 1005, 1002, 1006, 1203, 1207, 1204, 1208. This arrangement is made possible by the multiplicity of electrodes, which makes it possible to dedicate one electrode to high impedance while still having enough electrodes, of the right size, to carry out the other measurements. Arrangements 1300, 1400 illustrate cases where the passive electrode 1305, 1306, 1407, 1408 has the same size along the Y dimension as the active electrodes. In particular, the greater the number of independent electrodes per set LG, RG, the smaller the surface area of each electrode. Therefore, connecting a single electrode to the high impedance does not significantly reduce the contact area between the skin and the anode or cathode.

In arrangements with three or more independent electrodes per set of RG, LG electrodes (arrangements 1000, 1100, 1200, 1300a, 1300b, 1400), the electrode that is passive in DC configuration is positioned, along length Y, between two electrodes that are active in DC configuration. This positioning has two advantages: it increases the likelihood of the user touching the passive electrode (a shift of the foot along the length Y of the base 102 will have no consequence) and it limits the contact surface lost with the foot for the ESC: due to the curvature of the foot, contact between the electrode positioned under the arch of the foot is weaker than for the other electrodes. Consequently, the loss of surface area for ESC is lower than for other electrodes.

Alternatively, the passive electrode in DC configuration may be placed as an end electrode. This takes advantage of contact with the heel or ball of the foot to ensure that a user's body is set to high impedance.

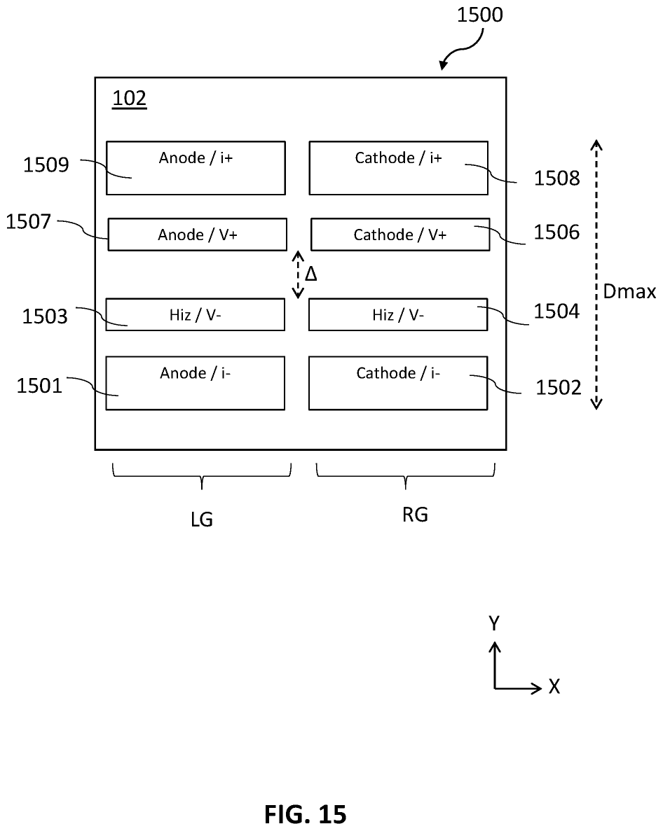

In all the arrangements described, the electrodes that are in the extreme position along the length Y may have a larger surface area than the other electrodes. Such an arrangement 1500 is illustrated in particular in FIG. 15, where the end electrodes (1501, 1502, 1508, 1509) may have a larger dimension along the length Y (e.g. twice as large) as the other electrodes (1503, 1504, 1506, 1507). When the electrodes have the same shape, both formulations are equivalent.

This means that on arrangements 1000, 1100, 1200, 1300a, 1300b, 1400, the end electrodes along length Y can have a larger dimension along length Y than that of the electrically conductive paths between the two end electrically conductive paths. For example, in FIG. 6, the strip pairs L1 and L3, L15 and L17, R2 and R4, R16 and R18 are not electrically independent, but are permanently connected at the electrical circuit level. This configuration makes it possible to increase the size of the end electrodes without disturbing the visual appearance of the upper face 604.

The electrodes can have a constant dimension along width X of base 102. This limits the sensitivity of measurements to the position of the foot along width X. This means that along width X, electrodes of the same RG, LG set start and end at the same point. Formulated differently, any straight line parallel to length Y passing through an electrically conductive path of a set passes through all the electrically conductive paths of the set. In an embodiment, on upper face 604, the electrodes each have a rectangular shape, with a dimension along width X greater than the dimension along length Y (ratio of at least five). On the upper side 604, the rectangles can have the same dimensions.

In an embodiment, the electrodes extend along the width X of the measuring station 100 over at least 40%, or even 45%, of the width X100 of the measuring station. This ensures that the foot makes contact with the electrodes, whatever its position along width X.

Having a regular arrangement of strips on the upper side can encourage the user to position himself between the end strips, and therefore allows the user to position himself naturally in the correct way. In addition, by having larger end electrodes, the likelihood of the foot touching both end electrodes is increased.

An identical strip layout ensures a regular appearance on the upper face, which helps to eliminate the “medical” effect of the measuring station 100 and thus enhances product retention (use of the station over time). In other words, the user has the impression of using a comfort product rather than a product for monitoring physiological parameters.

The distance Δ may be at least 8 cm, or even at least 9 cm. In particular, there is a constraint relating to the minimum length of a foot: if the distance Δ is too great, a small foot will not be able to be in contact with all four electrodes of the RG, LG set of electrodes (for an ACf configuration).

Between two successive ESCs, switch 566 or voltage generator 806 may invert the anode and cathode to regenerate the electrodes. This is because oxidation-reduction takes place at each ESC, degrading metal deposits or metal inserts.

In an embodiment, the measuring station 100 is used to take ECGs from the user. The sensors 104 used for this purpose are present on the substrate and on the handle, so that at least one foot and one hand are in contact with an electrode. In particular, both feet and both hands are in contact with at least one electrode.

Document WO2021/164561 describes an ECG scale with handle.

To obtain a multi-channel ECG, the left lower limb must be connected to a so-called LL (left leg) electrode. Thanks to switch 566, an ECG arrangement can include at least one electrode of the left LG set which is connected to ECG electrical circuit 568 (e.g. electrode L15/L17). Another arrangement further comprises an ACf configuration in the right foot, in order to perform an ECG at the same time as an IPG in the right foot. Alternatively, another arrangement further comprises an ACb configuration, except that only electrode L13 of electrodes L13, L15/L17 is connected to the alternating current generator (electrode L15/L17 being connected to electrode LL for ECG).

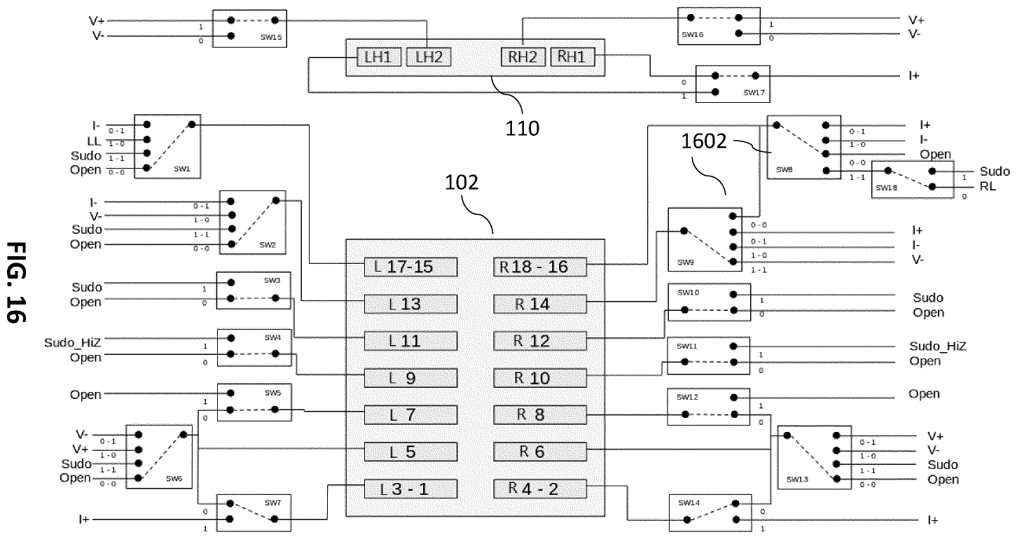

FIG. 16 shows a circuit diagram with the logic of switch 566 (with a simplified representation of the handle). Switch 566 comprises a plurality of switches, which are positioned, for example, on the PCB of base 102 and on the PCB of handle 110. The references on FIG. 16 are similar to those used previously (Sudo_HiZ being HiZ, Sudo being Cath or An, I being i, and open meaning that the switch is open). Each electrode L1 to R18, LH1, LH2, RH1, RH2 is connected to control circuitry 550 via one or more switches 1602. Each switch 1602 may be driven by one or more GPIO (“General Purpose Input/Output”) ports, represented by the values 0 and 1 next to the switch connections. A two-position switch is driven by a single GPIO, and a four-position switch is driven by two GPIOs. GPIO instructions are sent by the MCU, for example. In particular, switch 566 comprises at least one switch per electrode. In configurations with N independent electrodes (for an set of electrodes), the measuring station 100 comprises at least N switches.

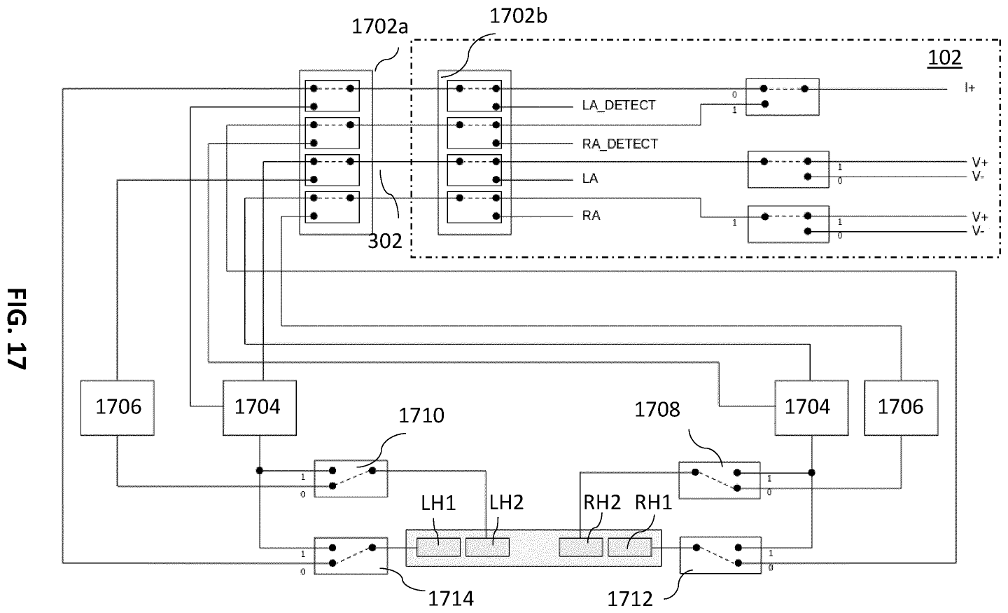

FIG. 17 shows the handle 110 in greater detail (with the exception of the dotted square representing the components in the base 102). The switch here comprises two master switches 1702a (in the handle), 1702b (in the base) facing each other are switches that comprise, for example, four switches that switch simultaneously in the same position. They enable switching between an ECG configuration and an impedance measurement configuration (BIA, IPG) by limiting the number of wires in cable 302 (to keep the cable cross-section small and flexible). Handle 110 comprises at least one handle ECG electrical circuit 1706 connected to at least one electrode LH1, LH2, RH1, RH2 and configured to process the signal before it transits through cable 302 (typically a follower circuit with an operational amplifier). Handle 110 comprises at least one electrical handle impedance measurement circuit 1706 connected to at least one electrode LH1, RH1 configured to process the signal before it transits through cable 302 (typically a follower circuit with an operational amplifier), and then to the voltmeter. The ECG and impedance measurement circuits are connected to master switch 1702a. LA_DETECT and RA_DETECT are part of a grab detection system on handle 110 which is not described in more detail here. The other switches on handle 110 connect electrodes RH1, RH2, LH1, LH2 to the ECG electrical circuit or to the BIA electrical circuit. In particular, switch 566 comprises a switch 1708 for selectively connecting electrode RH2 to ECG circuit 1704 or impedance measurement circuit 1706 (connected to the voltmeter); switch 566 comprises a switch 1710 for connecting electrode LH2 to ECG circuit 1704 or impedance measurement circuit 1706 (connected to the voltmeter); switch 566 comprises a switch 1712 for selectively connecting electrode RH1 to electrode RH2 or to an impedance measurement circuit (connected to the current source); switch 566 comprises a switch 1714 for selectively connecting electrode LH1 to electrode LH2 or to an impedance measurement circuit (connected to the current source). FIGS. 16 and 17 therefore fully describe the electronic configuration of switch 566 enabling the configurations described in the description to be implemented. The double switch 1702a, 1702b limits the number of conductors passing through cable 302. In particular, the handle comprises a master switch 1702 and each electrode of the handle is connected to a switch to toggle between an ECG processing circuit and an impedance measurement circuit.

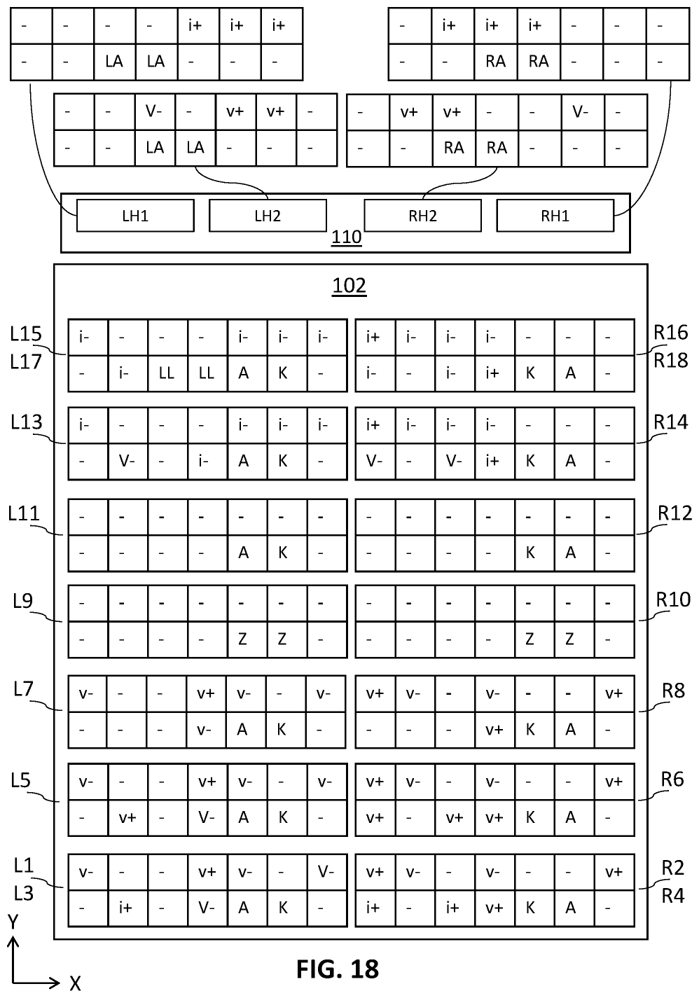

FIG. 18 schematically illustrates a set of electrode configurations for measuring station 100. The handle 110 and its four electrodes are shown; the base and its left and right sets LG, RG, with their seven electrodes each, are shown. Taking the electrical conduction paths 602 from FIG. 6, the seven electrodes of the left LG assembly are: L1 and L3 (which are linked), L5, L7, L9, 11, L13, L15 and L17 (which are linked); and the seven electrodes of the right assembly RG are: R2 and R4 (which are linked), R6, R8, R10, R12, R14, R16 and R18 (which are linked). In FIG. 18, thirteen configurations for electrodes' function are shown. The switch in FIG. 16 is used to toggle between the thirteen configurations. Each table comprises fourteen boxes (thirteen activated functions and one total disconnection), representing the electrodes' function for one configuration:

| | | | | | | |
|---|---|---|---|---|---|---|
| BIA or IPG | BIA / ICG | BIA (right | BIA (left | BIA /ICG | BIA (left | BIA (left leg) |
| (between | (right half - | arm) | leg) | (left half - | arm) | |
| the legs) | leg and | | | leg and | | |
| | arm) | | | arm) | | |
| IPG (in the | IPG (in the | ECG with | ECG with | ESC (anode | ESC (anode | Disconnection |
| right foot) | left foot) | IPG (in the | IPG | on left) | on right) | |
| | | right foot) | (between | | | |
| | | | the legs) | | | |

In FIG. 18, i+ and i− are the electrodes connected to the alternating current source (AC configuration); V+ and V− are the electrodes connected to the voltmeter (AC configuration); LL, LA, RA are the electrodes connected to the ECG acquisition system (ECG configuration); A and K are the electrodes connected to the direct voltage source (DC configuration); Z are the electrodes connected to the high impedance (DC configuration).

For each of the configurations shown in FIG. 17, a combination of GPIO ports is shown in FIG. 16, enabling the electrodes to be connected appropriately.

Alternatively, switch 566 can disconnect all electrodes from base 102.

In an embodiment, the electrodes are all made of ITO (indium-tin-oxide). The use of this material is documented for impedance measurement. However, for ESC, the material generally comprises steel or nickel. The inventors realized that ITO offered equivalent performance with superior deposition properties.

The measuring station 100 can perform a number of different measurements (ESC, BIA, IPG, etc.) by assigning different functions to the same electrically conductive paths. Switch 566 is used to toggle between configurations. The memory can store a program which comprises instructions which, when executed by the processor, enable different methods to be implemented.

According to an embodiment, the control circuitry 550 implements the following method: (E1) activation of switch 556 to put the electrodes in one configuration, (E2) triggering of a measurement associated with the configuration, (E3) activation of switch 556 to put the electrodes in another configuration, (E4) triggering of a measurement associated with the other configuration. The change of configuration may be any of the configurations described in the description and in particular in FIG. 18: DC to AC, including DC to ACf or DC to ACb. DC to ACf may be DC to ACf left foot or DC to ACf right foot, ACf to ACb, ACb to another ACb, etc.

1. A measuring station comprising:
a left set of electrodes, comprising at least two electrically independent electrodes arranged to contact the underside of a user's left foot,
a right set RD of electrodes, comprising at least two electrically independent electrodes arranged to contact the underside of a user's right foot,
a direct voltage source,
a switch configured to activate and deactivate a direct current, DC, configuration, in which at least one electrode of at least one of the left and the right set of electrodes is connected to the direct voltage source.

2. (canceled)

3. The measuring station according to claim 1, further comprising an alternating current source, and wherein:
the switch is configured to selectively activate or deactivate an alternating current, AC, configuration, in which at least one electrode of the electrodes of the left set and the right set of electrodes is connected to the alternating current source.

4. The measuring station according to claim 3, wherein, in AC configuration, the switch is configured to connect:
at least two electrodes of the electrodes of the left set of electrodes and the right set of electrodes to terminals of the alternating current source.

5. The measuring station according to claim 3, wherein, in AC configuration, the switch is configured to connect:
at least one electrode of the left set of electrodes and at least one electrode of the right set of electrodes to the terminals of alternating current source.

6. The measuring station according to claim 3, wherein, in AC configuration, the switch is configured to connect:
at least one electrode of a set of one of the right and left sets of electrodes to one terminal of the current source and another electrode of the same set to another terminal of the current source.

7. The measuring station according to claim 1, wherein:
the left set of electrodes and/or the right set of electrodes each comprise three electrically independent electrodes,
in DC configuration, the switch is configured to connect at least two electrodes of the left set of electrodes and/or at least two electrodes of the right set of electrodes to the direct voltage source.

8. The measuring station according to claim 1, wherein:
the left set of electrodes and/or the right set of electrodes each comprise four electrically independent electrodes, and
in DC configuration, the switch is configured to connect at least three electrodes of the left set of electrodes and/or at least three electrodes of the right set of electrodes to the direct voltage source.

9. (canceled)

10. (canceled)

11. The measuring station according to claim 1, wherein:
the left set of electrodes and/or the right set of electrodes each comprise at least seven electrically independent electrodes,
in DC configuration, the switch is configured to connect at least six electrodes of the left set of electrodes and/or at least six electrodes of the right set of electrodes to the direct voltage source.

12. The measuring station according to claim 1, wherein, in DC configuration, at least one passive electrode, of at least one of the right and left set of electrodes is not connected to the direct voltage source and wherein, in DC configuration, the passive electrode is located between two active electrodes.

13. The measuring station according to claim 1, wherein, in DC configuration, all the electrodes of the left set of electrodes and of the right set of electrodes are connected to the direct voltage source.

14. The measuring station according to claim 1, wherein, in DC configuration, one set of electrodes operates as cathode and one set of electrodes operates as anode.

15. (canceled)

16. (canceled)

17. (canceled)

18. The measuring station according to claim 1, wherein
the electrodes are spaced apart along a length of the measuring station, with the user's feet positioned along the length of the measuring station in normal use
and/or
the end electrodes along the length are wider.

19. The measuring station according to claim 1, wherein, in DC configuration at least one passive electrode of at least one of the right and left sets of electrodes is not connected to the direct voltage source and wherein, the electrodes configured to be active in DC configuration have a dimension in the direction of a length of the measuring station which is equal to or greater than that of the electrodes configured to be passive in DC configuration, the user's feet being positioned in normal use along the length of the measuring station.

20. The measuring station according to claim 1, wherein, for a set of electrodes, the electrodes configured to be active in DC configuration cover in surface at least 50% of a surface of a convex envelope defined by all the electrodes of the set, and/or a distance between the end electrodes, electrodes included, along a length of the measuring station is at least 20 cm, the user's feet positioning themselves in normal use along a length of the measuring station.

21. The measuring station according to claim 1, wherein the electrodes of the left set of electrodes extend along a width of the measuring station over more than 40% of the width of the measuring station and the electrodes of the right set of electrodes extend along a width of the measuring station over more than 40% of the width of the measuring station.

22. (canceled)

23. The measuring station according to claim 1, wherein the direct voltage source is configured to selectively apply successive steps of constant voltage to a pair of active electrodes, said electrodes of the pair of electrodes constituting an anode and a cathode.

24. The measuring station according to claim 23, wherein voltage values of successive steps are decreasing and/or last between 500 ms and 2 s each.

25. The measuring station according to claim 1, further comprising a weight sensor.

26. The measuring station according to claim 1, comprising:
a base comprising the left set of electrodes and the right set of electrodes,
a handle with at least one electrode suitable for contact with the hand, in which, in DC configuration, the switch is configured to connect all the base electrodes to terminals of the direct voltage source and to connect the handle electrode to a high impedance.

27. (canceled)

28. A measurement method using the measuring station according to claim 1, comprising a step of switching, using the switch, between two different configurations.

29.-32. (canceled)
